(19) United States
(12) Patent Application Publication
(10) Pub. No.: US 2020/0058375 A1
(43) Pub. Date: Feb. 20, 2020

(54) VARIANT-SPECIFIC ALIGNMENT OF NUCLEIC ACID SEQUENCING DATA

(71) Applicants: Jay DUNFFNER, Cambridge, MA (US); Momenta Pharmaceuticals, Inc., Cambridge, MA (US)

(72) Inventor: Jay Duffner, Cambridge, MA (US)

(21) Appl. No.: 16/487,339

(22) PCT Filed: Feb. 23, 2018
(86) PCT No.: PCT/US2018/019383

§ 371 Date: Aug. 20, 2019

Related U.S. Application Data
(60) Provisional application No. 62/462,681, filed on Feb. 23, 2017.

(51) Int. Cl. G16B 30/10; G16B 30/20; G16B 20/00

(52) U.S. Cl. CPC G16B 30/10; G16B 20/00; G16B 30/20

(57) ABSTRACT

Techniques and systems for determining a correct alignment of nucleic acid sequences are described. Determining the correct alignment may include generating multiple reference sequences that include one or more variants and aligning the nucleic acid sequences to the multiple reference sequences. The correct alignment may include performing an alignment of the nucleic acid sequences using the multiple reference sequences and determining the correct alignment for the nucleic acid sequences based at least in part on a result of the alignment using the multiple reference sequences.

### TECHNICAL FIELD

Described herein are embodiments of systems and related methods for analyzing nucleic acid sequencing data. In some embodiments, the analysis of nucleic acid sequencing data can include alignment of sequence reads to multiple different reference sequences to identify information related to where the sequence reads align. This may be used in some embodiments to identify the region, of a set of homologous regions, to which the sequence reads align or to identify a variant, of a set of variants for a region, to which the sequence reads align. In some embodiments, the techniques may be used to identify, for a set of homologous regions that are each associated with variants, the variant and the homologous region to which sequence reads align.

### BACKGROUND

Nucleic acid sequencing techniques may determine an arrangement of nucleotides within a nucleic acid, such as a deoxyribose nucleic acid (DNA) or a ribonucleic acid (RNA). Sequencing data from nucleic acid sequencing technologies (e.g., Sanger-type sequencers, Next Generation Sequencing (NGS) technologies, or others) can include information identifying nucleotide sequences for fragments of nucleic acid sequences complementary to a target nucleic acid sequence. Sequencing data from a sequencer can include series of nucleotides corresponding to these fragments and may be referred to as sequence reads. Each sequence read may identify a number of nucleotides in a nucleic acid sequence, determined by the sequencer for a sample.

Analysis of sequencing data may provide insights into the genome of a particular individual. Since some regions of the genome may vary across different individuals, determining an individual's unique genomic variation can have implications for understanding the individual's health and genetic predisposition to certain diseases, which may provide information to develop health care personalized to the individual. Analysis of sequencing data may include performing an alignment process that determines where particular sequence reads map to a reference sequence to identify regions of the reference sequence that are similar to the sequence reads. The reference sequence may be for a type of organism. Alignment of the sequence reads to a reference sequence may allow for identification of genomic locations within the reference sequence for the type of organism that the sequence reads map to.

When performing alignment, some sequence reads may precisely match to a series of nucleotides of a reference sequence. In other cases, though, sequence reads may closely, but not precisely, map to a region of the reference sequence. This may be due, for example, to errors in determining sequence reads from a sample, but may also be due to normal variation between organisms in their nucleic acids (e.g., DNA). In some cases, a reference sequence used in alignment may correspond to a scientific consensus on a “standard” or “average” nucleic acid sequence for a species, or for a gene, but because individual organisms have varying DNA, the sequence reads may not precisely align to that average.

Accordingly, in some cases of alignment, many of the nucleotides of sequence reads may precisely match a region of the reference sequence at a number of nucleotide positions within the reference sequence, but alongside the matching sequence reads a number of other sequence reads may differ from the region in a type of nucleotide (e.g., A, T, C, G) at one or more nucleotide positions.

In cases where sequence reads do not precisely match a reference sequence, there can be uncertainty in alignment that can generate errors in subsequent analysis of the sequencing data. The errors may include incorrectly identifying a location of the reference sequence to which a particular sequence read aligns. Because an alignment may be used as a diagnostic tool, such as by determining whether a particular gene is present, an incorrect alignment may have significant repercussions. For example, further analysis of the sequence reads following the mis-alignment may incorrectly identify a characteristic of an organism associated with the sequence reads because of that incorrect alignment. This may include an organism being incorrectly genotyped because of an incorrect alignment of a sequence read to a reference sequence, such as by incorrectly identifying an organism as having a particular gene or gene variant, which may in turn lead to incorrectly identifying an organism (which may include a human patient) as having or being at increased risk for having a particular medical condition.

### SUMMARY

According to an aspect of the present application, a method of analyzing sequencing data is provided, the method comprising determining a correct alignment of a plurality of nucleic acid sequences. The correct alignment is a target region having a first series of nucleotides at a first sequence location or at least one non-target region having at least one second series of nucleotides at at least one second sequence location. Determining the correct alignment comprises determining at least one target region variant for the first series of nucleotides and at least one non-target region variant for the at least one second series of nucleotides. Each of the at least one target region variant includes at least one variation from the first series of nucleotides and each of the at least one non-target region variant includes at least one variation from one of the at least one second series of nucleotides. Determining the correct alignment further comprises generating a plurality of reference nucleic acid sequences based on the at least one target region variant and the at least one non-target region variant, performing an alignment of the plurality of nucleic acid sequences using the plurality of reference nucleic acid sequences, and determining the correct alignment for the plurality of nucleic acid sequences based at least in part on a result of the alignment using the plurality of reference nucleic acid sequences.

According to an aspect of the present application at least one computer-readable storage medium storing computer-executable instructions that, when executed, perform a method of analyzing sequence data is provided, the method comprising determining a correct alignment of a plurality of nucleic acid sequences. The correct alignment is a target region having a first series of nucleotides at a first sequence location or at least one non-target region having at least one second series of nucleotides at at least one second sequence location. Determining the correct alignment comprises determining at least one target region variant for the first series of nucleotides and at least one non-target region variant for the at least one second series of nucleotides. Each of the at least one target region variant includes at least one variation from the first series of nucleotides and each of the at least one non-target region variant includes at least one variation from one of the at least one second series of nucleotides. Determining the correct alignment further comprises generating a plurality of reference nucleic acid sequences based on the at least one target region variant and the at least one non-target region variant, performing an alignment of the plurality of nucleic acid sequences using the plurality of reference nucleic acid sequences, and determining the correct alignment for the plurality of nucleic acid sequences based at least in part on a result of the alignment using the plurality of reference nucleic acid sequences.

According to an aspect of the present application, an apparatus is provided, the apparatus comprising control circuitry configured to determine a correct alignment of a plurality of nucleic acid sequences. The correct alignment is a target region having a first series of nucleotides at a first sequence location or at least one non-target region having at least one second series of nucleotides at at least one second sequence location. Determining the correct alignment comprises determining at least one target region variant for the first series of nucleotides and at least one non-target region variant for the at least one second series of nucleotides. Each of the at least one target region variant includes at least one variation from the first series of nucleotides and each of the at least one non-target region variant includes at least one variation from one of the at least one second series of nucleotides. Determining the correct alignment further comprises generating a plurality of reference nucleic acid sequences based on the at least one target region variant and the at least one non-target region variant, performing an alignment of the plurality of nucleic acid sequences using the plurality of reference nucleic acid sequences, and determining the correct alignment for the plurality of nucleic acid sequences based at least in part on a result of the alignment using the plurality of reference nucleic acid sequences.

According to an aspect of the present application, a method for genotyping an individual is provided, the method comprising determining a genotype for the individual from a plurality of nucleic acid sequences associated with the individual. The genotype is based on a first gene at a first sequence location or a second gene at a second sequence location. Determining the genotype comprises determining at least one first gene variant for a first series of nucleotides associated with the first gene and at least one second gene variant for a second series of nucleotides associated with the second gene. The first series of nucleotides includes at least one variation from the second series of nucleotides. Each of the at least one first gene variant includes at least one variation from the first series of nucleotides and each of the at least one second gene variant includes at least one variation from one of the second series of nucleotides. Determining the genotype further comprises generating a plurality of reference nucleic acid sequences based on the at least one first gene variant and the at least one second gene variant, performing an alignment of the plurality of nucleic acid sequences using the plurality of reference nucleic acid sequences, and determining the genotype for the individual based at least in part on a result of the alignment using the plurality of reference nucleic acid sequences to identify the first series of nucleotides or one of the at least one first gene variant as being present at the first location and/or the second series of nucleotides or one of the at least one second gene variant as being present at the second location.

In one aspect, the present disclosure provides for a system comprising, consisting of, or consisting essentially of: a nucleic acid sequencer; a nucleic acid analysis device; an alignment device, the alignment device configured to: receive a plurality of nucleic acid sequences from the nucleic acid sequencer; determine a correct alignment of a plurality of nucleic acid sequences, wherein the correct alignment is a target region having a first series of nucleotides at a first sequence location or at least one non-target region having at least one second series of nucleotides at least one second sequence location, wherein determining the correct alignment comprises, consists of, or consists essentially of: determining at least one target region variant for the first series of nucleotides and at least one non-target region variant for the at least one second series of nucleotides, wherein each of the at least one target region variant includes at least one variation from the first series of nucleotides and each of the at least one non-target region variant includes at least one variation from one of the at least one second series of nucleotides, generating a plurality of reference nucleic acid sequences based on the at least one target region variant and the at least one non-target region variant, performing an alignment of the plurality of nucleic acid sequences using the plurality of reference nucleic acid sequences, and determining the correct alignment for the plurality of nucleic acid sequences based at least in part on a result of the alignment using the plurality of reference nucleic acid sequences; and provide the correct alignment for the plurality of nucleic acid sequences to the nucleic acid alignment device.

In some embodiments of the systems described herein, generating the plurality of reference nucleic acid sequences comprises, consists of, or consists essentially of generating a plurality of reference nucleic acid sequences from the first series of nucleotides for the target region, the at least one target region variant, the at least one second series of nucleotides for the at least one non-target region, and the at least one non-target region variant.

In some embodiments of the systems described herein, the at least one second sequence location is a second sequence location; the at least one second series of nucleotides is a second series of nucleotides; and generating the plurality of reference nucleic acid sequences comprises, consists of, or consists essentially of generating a plurality of sequences including, at the first sequence location, one of the first series of nucleotides or the at least one target region variant and including, at the second sequence location, one of the second series of nucleotides or the at least one non-target region variant, each sequence of the plurality of sequences being different.

In some embodiments of the systems described herein, determining the correct alignment further comprises, consists of, or consists essentially of: determining the at least one non-target region, wherein determining the at least one non-target region comprises, consists of, or consists essentially of analyzing an alignment of at least a subset of the plurality of nucleic acid sequences to a reference sequence to identify regions to which the at least the subset align.

In some embodiments of the systems described herein, generating the plurality of reference nucleic acid sequences comprises, consists of, or consists essentially of generating a first reference nucleic acid sequence, of the plurality, by modifying the reference sequence at the first sequence location to substitute one target region variant, of the at least one target region variant, for the target region of the reference sequence. In some embodiments of the systems described herein, generating the plurality of reference nucleic acid sequences comprises, consists of, or consists essentially of generating a second reference nucleic acid sequence, of the plurality, by modifying the reference sequence at the second sequence location to substitute one non-target region variant, of the at least one non-target region variant, for the non-target region of the reference sequence. In some embodiments of the systems described herein, the plurality of nucleic acid sequences comprise, consist of, or consist essentially of human DNA and the reference sequence is a human genome sequence.

In some embodiments of the systems described herein, the alignment device is further configured to: determine the at least one non-target region based on the target region, wherein determining the at least one non-target region comprises, consists of, or consists essentially of identifying one or more regions of a genome that are homologous with the target region.

In some embodiments of the systems described herein, identifying one or more regions of a genome that are homologous with the target region comprises, consists of, or consists essentially of identifying one or more regions of a genome that have a degree of similarity to the target region above a threshold. In some embodiments of the systems described herein, identifying one or more regions of a genome that have a degree of similarity to the target region above a threshold comprises, consists of, or consists essentially of identifying one or more regions of the genome that have a degree of similarity to the target region that is higher than a degree of inter-organism variability for the target region.

In some embodiments of the systems described herein, determining the correct alignment comprises, consists of, or consists essentially of: determining a first nucleic acid sequence of the plurality of nucleic acid sequences that aligns to a first reference sequence of the plurality of reference sequences at least at the first sequence location, identifying the first nucleic acid sequence as having a target region variant of the at least one target region variant at the first sequence location of the first reference sequence, and outputting an indication that the first nucleic acid sequence includes the target region variant.

In some embodiments of the systems described herein, the alignment device is further configured to determine an amino acid sequence associated with the first nucleic acid sequence based on a nucleic acid sequence for the target region variant.

In some embodiments of the systems described herein, outputting the indication that the first nucleic acid sequence includes the target region variant comprises outputting an indication of the amino acid sequence. In some embodiments of the systems described herein, outputting the indication that the first nucleic acid sequence includes the target region variant comprises outputting an indication of a protein associated with the amino acid sequence.

In some embodiments of the systems described herein, the method further comprises, consists of, or consists essentially of determining a first amino acid sequence associated with a first target region variant at the first location of the first reference sequence and a second amino acid sequence associated with a second target region variant at the first location of the second reference sequence.

In some embodiments of the systems described herein, determining the correct alignment comprises, consists of, or consists essentially of determining a first portion of the plurality of nucleic acid sequences that align to a first reference sequence of the plurality of reference sequences and determining a second portion of the plurality of nucleic acid sequences that align to a second reference sequence of the plurality of reference sequences. In some embodiments of the systems described herein, determining the correct alignment comprises, consists of, or consists essentially of determining an amount of nucleic acid sequences of the plurality of nucleic acid sequences that align with each of the plurality of reference sequences. In some embodiments of the systems described herein, determining the correct alignment comprises, consists of, or consists essentially of determining a reference sequence of the plurality of reference sequences that the nucleic acid sequence aligns to and identifying a series of nucleotides at the first location in the reference sequence; and the nucleic acid analysis device is configured to assign a genotype for an individual associated with a nucleic acid sequence of the plurality of nucleic acid sequences based on the reference sequence of the plurality of reference sequences to which the nucleic acid sequence aligns.

In some embodiments of the systems described herein, the at least one target region variant includes a plurality of target region variants and the at least one non-target region variant includes a plurality of non-target region variants, and generating the plurality of reference nucleic acid sequences further comprises generating the plurality of reference nucleic acid sequences to have all unique combinations of the plurality of target region variants at the first sequence location and the plurality of non-target region variants at the second sequence location. In some embodiments of the systems described herein, the target region includes at least a portion of a first gene and the non-target region includes at least a portion of a second gene. In some embodiments of the systems described herein, the sequence data is human DNA sequence data, the at least one target region includes a nucleotide coding sequence for a FC-receptor, and the at least one non-target region includes a nucleotide sequence homologous to the nucleotide coding sequence. In some embodiments of the systems described herein, the FC-receptor is selected from the group consisting of FCGR1A, FCGR1B, FCGR1C, FCGR2A, FCGR2B, FCGR2C, FCGR3A, and FCGR3B. In some embodiments of the systems described herein, the method further comprises, consists of, or consists essentially of identifying a first nucleic acid sequence of the plurality of nucleic acid sequences corresponding to FCGR3A and a second nucleic acid sequence of the plurality of nucleic acid sequences corresponding to FCGR3B.

In some embodiments of the systems described herein, the nucleic acid sequencer is coupled to the alignment device, and the alignment device is coupled to the nucleic acid analysis device.

In some embodiments of the systems described herein, identifying the non-target region having the second series of nucleotides at the second sequence location further comprises, consists of, or consists essentially of identifying the second series of nucleotides as having at least one single-nucleotide polymorphism in comparison to the first series of nucleotides at the first location.

In some embodiments of the systems described herein, the nucleic acid analysis device is configured to: determine a genotype for the individual from the plurality of nucleic acid sequences, wherein the plurality of nucleic acid sequences are associated with the individual. In some embodiments of the systems described herein, the nucleic acid analysis device is further configured to determine an amino acid sequence based on the identified variant. In some embodiments of the systems described herein, the nucleic acid analysis device is further configured to determine a protein structure based on the amino acid sequence. In some embodiments of the systems described herein, the nucleic acid analysis device is further configured to: determine a genotype for a second individual by performing an alignment of a second plurality of nucleic acid sequences associated with the second individual using the plurality of reference nucleic acid sequences to identify the first series of nucleotides or one of the at least one first gene variant as being present at the first location and/or the second series of nucleotides or one of the at least one second gene variant as being present at the second location.

In another aspect, the present disclosure provides for a method of analyzing sequencing data, the method comprising, consisting of, or consisting essentially of: determining a correct alignment of a plurality of nucleic acid sequences, wherein the correct alignment is a target region having a first series of nucleotides at a first sequence location or at least one non-target region having at least one second series of nucleotides at at least one second sequence location, wherein determining the correct alignment comprises, consists of, or consists essentially of: determining at least one target region variant for the first series of nucleotides and at least one non-target region variant for the at least one second series of nucleotides, wherein each of the at least one target region variant includes at least one variation from the first series of nucleotides and each of the at least one non-target region variant includes at least one variation from one of the at least one second series of nucleotides; generating a plurality of reference nucleic acid sequences based on the at least one target region variant and the at least one non-target region variant; performing an alignment of the plurality of nucleic acid sequences using the plurality of reference nucleic acid sequences; and determining the correct alignment for the plurality of nucleic acid sequences based at least in part on a result of the alignment using the plurality of reference nucleic acid sequences.

In some embodiments of the methods described herein, generating the plurality of reference nucleic acid sequences comprises, consists of, or consists essentially of generating a plurality of reference nucleic acid sequences from the first series of nucleotides for the target region, the at least one target region variant, the at least one second series of nucleotides for the at least one non-target region, and the at least one non-target region variant.

In some embodiments of the methods described herein, the at least one second sequence location is a second sequence location; the at least one second series of nucleotides is a second series of nucleotides; and generating the plurality of reference nucleic acid sequences comprises, consists of, or consists essentially of generating a plurality of sequences including, at the first sequence location, one of the first series of nucleotides or the at least one target region variant and including, at the second sequence location, one of the second series of nucleotides or the at least one non-target region variant, each sequence of the plurality of sequences being different.

In some embodiments of the methods described herein, determining the correct alignment further comprises, consists of, or consists essentially of: determining the at least one non-target region, wherein determining the at least one non-target region comprises, consists of, or consists essentially of analyzing an alignment of at least a subset of the plurality of nucleic acid sequences to a reference sequence to identify regions to which the at least the subset align.

In some embodiments of the methods described herein, generating the plurality of reference nucleic acid sequences comprises, consists of, or consists essentially of generating a first reference nucleic acid sequence, of the plurality, by modifying the reference sequence at the first sequence location to substitute one target region variant, of the at least one target region variant, for the target region of the reference sequence. In some embodiments of the methods described herein, generating the plurality of reference nucleic acid sequences comprises, consists of, or consists essentially of generating a second reference nucleic acid sequence, of the plurality, by modifying the reference sequence at the second sequence location to substitute one non-target region variant, of the at least one non-target region variant, for the non-target region of the reference sequence. In some embodiments of the methods described herein, the plurality of nucleic acid sequences comprise, consist of, or consist essentially of human DNA and the reference sequence is a human genome sequence.

In some embodiments, the methods described herein further comprise, consist of, or consist essentially of: determining the at least one non-target region based on the target region, wherein determining the at least one non-target region comprises, consists of, or consists essentially of identifying one or more regions of a genome that are homologous with the target region.

In some embodiments of the methods described herein, identifying one or more regions of a genome that are homologous with the target region comprises, consists of, or consists essentially of identifying one or more regions of a genome that have a degree of similarity to the target region above a threshold. In some embodiments of the methods described herein, identifying one or more regions of a genome that have a degree of similarity to the target region above a threshold comprises, consists of, or consists essentially of identifying one or more regions of the genome that have a degree of similarity to the target region that is higher than a degree of inter-organism variability for the target region.

In some embodiments of the methods described herein, determining the correct alignment comprises, consists of, or consists essentially of: determining a first nucleic acid sequence of the plurality of nucleic acid sequences that aligns to a first reference sequence of the plurality of reference sequences at least at the first sequence location, identifying the first nucleic acid sequence as having a target region variant of the at least one target region variant at the first sequence location of the first reference sequence, and outputting an indication that the first nucleic acid sequence includes the target region variant.

In some embodiments of the methods described herein, the method further comprises, consists of, or consists essentially of determining an amino acid sequence associated with the first nucleic acid sequence based on a nucleic acid sequence for the target region variant. In some embodiments of the methods described herein, outputting the indication that the first nucleic acid sequence includes the target region variant comprises outputting an indication of the amino acid sequence. In some embodiments of the methods described herein, outputting the indication that the first nucleic acid sequence includes the target region variant comprises outputting an indication of a protein associated with the amino acid sequence.

In some embodiments of the methods described herein, determining the correct alignment comprises, consists of, or consists essentially of determining a first portion of the plurality of nucleic acid sequences that align to a first reference sequence of the plurality of reference sequences and determining a second portion of the plurality of nucleic acid sequences that align to a second reference sequence of the plurality of reference sequences.

In some embodiments of the methods described herein, the method further comprises, consists of, or consists essentially of determining a first amino acid sequence associated with a first target region variant at the first location of the first reference sequence and a second amino acid sequence associated with a second target region variant at the first location of the second reference sequence. In some embodiments of the methods described herein, determining the correct alignment comprises, consists of, or consists essentially of determining an amount of nucleic acid sequences of the plurality of nucleic acid sequences that align with each of the plurality of reference sequences.

In some embodiments of the methods described herein, determining the correct alignment comprises, consists of, or consists essentially of determining a reference sequence of the plurality of reference sequences that the nucleic acid sequence aligns to and identifying a series of nucleotides at the first location in the reference sequence; and the method further comprises, consists of, or consists essentially of assigning a genotype for an individual associated with a nucleic acid sequence of the plurality of nucleic acid sequences based on the reference sequence of the plurality of reference sequences to which the nucleic acid sequence aligns.

In some embodiments of the methods described herein, the at least one target region variant includes a plurality of target region variants and the at least one non-target region variant includes a plurality of non-target region variants, and generating the plurality of reference nucleic acid sequences further comprises, consists of, or consists essentially of generating the plurality of reference nucleic acid sequences to have all unique combinations of the plurality of target region variants at the first sequence location and the plurality of non-target region variants at the second sequence location.

In some embodiments of the methods described herein, the target region includes at least a portion of a first gene and the non-target region includes at least a portion of a second gene. In some embodiments of the methods described herein, the sequence data is human DNA sequence data, the at least one target region includes a nucleotide coding sequence for a FC-receptor, and the at least one non-target region includes a nucleotide sequence homologous to the nucleotide coding sequence. In some embodiments of the methods described herein, the FC-receptor is selected from the group consisting of FCGR1A, FCGR1B, FCGR1C, FCGR2A, FCGR2B, FCGR2C, FCGR3A, and FCGR3B. In some embodiments of the methods described herein, the method further comprises, consists of, or consists essentially of identifying a first nucleic acid sequence of the plurality of nucleic acid sequences corresponding to FCGR3A and a second nucleic acid sequence of the plurality of nucleic acid sequences corresponding to FCGR3B.

In some embodiments of the methods described herein, identifying the non-target region having the second series of nucleotides at the second sequence location further comprises identifying the second series of nucleotides as having at least one single-nucleotide polymorphism in comparison to the first series of nucleotides at the first location.

In another aspect, the present disclosure provides for at least one computer-readable storage medium storing computer-executable instructions that, when executed, perform a method of analyzing sequence data, the method comprising, consisting of, or consisting essentially of: determining a correct alignment of a plurality of nucleic acid sequences, wherein the correct alignment is a target region having a first series of nucleotides at a first sequence location or at least one non-target region having at least one second series of nucleotides at at least one second sequence location, wherein determining the correct alignment comprises, consists of, or consists essentially of: determining at least one target region variant for the first series of nucleotides and at least one non-target region variant for the at least one second series of nucleotides, wherein each of the at least one target region variant includes at least one variation from the first series of nucleotides and each of the at least one non-target region variant includes at least one variation from one of the at least one second series of nucleotides; generating a plurality of reference nucleic acid sequences based on the at least one target region variant and the at least one non-target region variant; performing an alignment of the plurality of nucleic acid sequences using the plurality of reference nucleic acid sequences; and determining the correct alignment for the plurality of nucleic acid sequences based at least in part on a result of the alignment using the plurality of reference nucleic acid sequences.

In another aspect, the present disclosure provides for an apparatus comprising, consisting of, or consisting essentially of: control circuitry configured to: determine a correct alignment of a plurality of nucleic acid sequences, wherein the correct alignment is a target region having a first series of nucleotides at a first sequence location or at least one non-target region having at least one second series of nucleotides at at least one second sequence location, wherein determining the correct alignment comprises, consists of, or consists essentially of: determining at least one target region variant for the first series of nucleotides and at least one non-target region variant for the at least one second series of nucleotides, wherein each of the at least one target region variant includes at least one variation from the first series of nucleotides and each of the at least one non-target region variant includes at least one variation from one of the at least one second series of nucleotides; generating a plurality of reference nucleic acid sequences based on the at least one target region variant and the at least one non-target region variant; performing an alignment of the plurality of nucleic acid sequences using the plurality of reference nucleic acid sequences; and determining the correct alignment for the plurality of nucleic acid sequences based at least in part on a result of the alignment using the plurality of reference nucleic acid sequences.

In another aspect, the present disclosure provides for a method for genotyping an individual, the method comprising, consisting of, or consisting essentially of: determining a genotype for the individual from a plurality of nucleic acid sequences associated with the individual, wherein the genotype is based on a first gene at a first sequence location or a second gene at a second sequence location, and wherein determining the genotype comprises, consists of, or consists essentially of: determining at least one first gene variant for a first series of nucleotides associated with the first gene and at least one second gene variant for a second series of nucleotides associated with the second gene, wherein the first series of nucleotides includes at least one variation from the second series of nucleotides, and wherein each of the at least one first gene variant includes at least one variation from the first series of nucleotides and each of the at least one second gene variant includes at least one variation from one of the second series of nucleotides; generating a plurality of reference nucleic acid sequences based on the at least one first gene variant and the at least one second gene variant; performing an alignment of the plurality of nucleic acid sequences using the plurality of reference nucleic acid sequences; and determining the genotype for the individual based at least in part on a result of the alignment using the plurality of reference nucleic acid sequences to identify the first series of nucleotides or one of the at least one first gene variant as being present at the first location and/or the second series of nucleotides or one of the at least one second gene variant as being present at the second location.

In some embodiments of the methods described herein, the method further comprises, consists of, or consists essentially of determining an amino acid sequence based on the identified variant. In some embodiments of the methods described herein, the method further comprises, consists of, or consists essentially of determining a protein structure based on the amino acid sequence.

In some embodiments of the methods described herein, the method further comprises, consists of, or consists essentially of: determining a genotype for a second individual by performing an alignment of a second plurality of nucleic acid sequences associated with the second individual using the plurality of reference nucleic acid sequences to identify the first series of nucleotides or one of the at least one first gene variant as being present at the first location and/or the second series of nucleotides or one of the at least one second gene variant as being present at the second location.

### BRIEF DESCRIPTION OF THE DRAWINGS

Various aspects and embodiments of the application will be described with reference to the following figures. It should be appreciated that the figures are not necessarily drawn to scale. Items appearing in multiple figures are indicated by the same reference number in all the figures in which they appear.

FIG. 1 illustrates components of an exemplary system that performs nucleic acid sequencing of a biological sample and analyzes the sequencing results.

FIG. 2 is a flowchart illustrating an exemplary method for analyzing sequencing data by aligning sequence reads to multiple reference sequences.

FIG. 3 is a flowchart illustrating an exemplary method for analyzing sequencing data by aligning the sequencing data to multiple reference sequences generated based on target region variant(s) and non-target region variant(s).

FIG. 4 is a flowchart illustrating an exemplary method for genotyping an individual by analyzing sequencing data associated with the individual.

FIG. 5 is a flowchart illustrating an exemplary method for analyzing sequencing data by using either a single reference sequence or by generating multiple reference sequences.

FIG. 6 is a flowchart illustrating an exemplary method for identifying variant(s) for a target region and/or for a non-target region.

FIG. 7 is a flowchart illustrating an exemplary method for modifying a reference sequence to include a variant for a target region and/or a variant for a non-target region.

FIG. 8 is a flowchart illustrating an exemplary method for identifying a variant as being present in a sequence read.

FIG. 9 is a block diagram of a computing device with which some embodiments may operate.

### DETAILED DESCRIPTION

Described herein are techniques for performing alignment of sequence reads of nucleic acid sequences using multiple different reference sequences in the alignment. In some embodiments, an alignment is performed to determine whether the sequence reads match a target region of a reference sequence, where the target region may be, for example, a particular gene and the sequence reads may have been generated in part using (in an assay) a primer that is intended to amplify nucleic acid sequences matching that target region. More specifically, in some embodiments, rather than only determining whether the sequence reads align to a single target region using a reference sequence for that target region, it may be determined whether the sequence reads align to the target region or to one or more other non-target regions. Additionally or alternatively, it may be determined whether the sequence reads align to one or more known variants for the target region and/or for the non-target region. Accordingly, in some embodiments, prior to alignment, multiple different reference sequences may be determined that represent a target region, one or more non-target regions, one or more variants of the target region and non-target region(s), and/or different combinations thereof. The alignment may then be performed on the nucleic acid sequences using the multiple different reference sequences and a matching alignment for the nucleic acid sequences may be determined based on a result of the alignment of multiple sequence reads to the multiple reference sequences. In some embodiments, the multiple sequence reads may have been determined from a single sample or samples for a single organism, and a single matching alignment for the sample or organism may be determined from the alignment of the multiple sequence reads.

The inventors recognized and appreciated the desirability of techniques for analyzing nucleic acid sequencing data that may aid in mitigating or eliminating potential uncertainty in alignment of sequence reads to a reference sequence. In some cases, a non-target region may be a potential source of uncertainty in alignment, as the non-target region may be homologous to the target region. As discussed below, homologous regions may include similar nucleotides. In such cases where the non-target region is homologous, there may be a high likelihood that a sequence read may incorrectly align to the non-target region using some alignment processes and/or may introduce an ambiguity as to the correct alignment.

The inventors also recognized and appreciated that uncertainty in alignment may also arise from one or more variants for a target region and/or a non-target region. A variant may include one or more nucleotide variations from a particular series of nucleotides. Variants may include, for example, different nucleotide sequences of a particular gene, such as a “standard” or “common” sequence for a species and another sequence of that gene that may be associated with a particular physical characteristic, a risk of a medical condition, etc. Such variants may be very similar to one another, and it may be difficult in some such cases to differentiate between variants when performing alignment.

The inventors further recognized and appreciated that difficulties with uncertainty in alignment may be particularly acute where a target region is homologous with a non-target region and in which there are known variants for both the target region and the non-target region. In some particularly difficult cases, because the target and non-target regions are homologous, some of the variants for the target region may be more similar to variants of the other non-target region than they are to other variants of the target region. In other words, in some cases, the degree of variation in a target region may be greater than the degree of similarity of the target region (and/or one or more variants thereof) to the non-target region (and/or one or more variants thereof). This may make achieving certainty in alignment particularly difficult in some cases, including because it may be difficult to make an alignment process sufficiently “fuzzy” to align a sequence read to the variants of a target region without inadvertently also aligning the variants of the non-target region.

As mentioned above, in some cases a sequence read may align precisely to a region of a reference genome due to a precise match between the nucleotides of the sequence read and of that region of the reference genome. However, this may not be common. Due to variation that may arise in nucleic acids between organisms of a species, or even in some cases between cells of an organism, it may often be the case that a sequence read that correctly matches to a region of a reference genome does not precisely align and that there are variations between nucleotides of the sequence read and the reference genome. Some alignment techniques have been proposed to remedy this difficulty by enabling “fuzzy” matching, where a sequence read may be identified as matching when it does not precisely match, but has a number of matching nucleotides above a threshold, or a number of differences or is otherwise determined to be a “close” match.

The inventors have recognized and appreciated, however, that these alignment techniques that provide for “fuzzy” matching may trigger additional difficulties in alignment of homologous regions with known variants. Regions of nucleotides can be homologous in terms of type of nucleotide (e.g., A, T, C, G) and position of nucleotides. A region may be considered homologous with another region when the two regions have a level of similarity in the series of nucleotides at the two regions. The homologous regions can have one or more nucleotide variations within the series of nucleotides. In some instances, two regions may be considered homologous when there is at least 80%, at least 90%, or at least 95% similarity between the two regions. Homologous regions may present particular challenges in alignment and sequencing, because the similarity may make it different to accurately determine whether a particular sequence read aligns with one region or with another homologous region. In some cases, a high precision is required to resolve ambiguity in the match between one region or the other and prevent misalignment of a sequence read and incorrect identification of a location within the reference sequence associated with a particular sequence read. The inventors have recognized and appreciated, however, that there is a tension between the advantages of “fuzzy” matching to permit for variation in genomic regions and of precise matching to disambiguate homologous regions. This tension may be greatest in regions that are both homologous and have a high degree of variation. In some such cases, the degree of variation may be greater than the degree of homology. In such a case, some variants of a gene may be more similar to a reference sequence for a homologous gene, which can lead to an unacceptably high likelihood of incorrect alignment of sequence reads for that gene, and an undesirably low confidence of a correct match.

As an example, developing primer targets for FC-receptor genes (e.g., FCGR1A, FCGR1B, FCGR1C, FCGR2A, FCGR2B, FCGR2C, FCGR3A, FCGR3B) can be difficult because of the high homology among some sets of these genes. The genes FCGR3A and FCGR3B can be approximately 96.8% identical at the nucleotide level, which makes it challenging to determine unique primers for these two genes that would amplify one of these genes preferentially over the other during sequencing. In addition, FCGR3B has at least six variants that arise from polymorphisms and FCGR3A has at least one variant. Some of the FCGR3B variants may be more similar to a reference sequence for FCGR3A than for a reference sequence for FCGR3B. This may lead, during alignment, to some of the FCGR3B variants incorrectly aligning to the reference sequence for FCGR3A rather than correctly aligning to the reference sequence for FCGR3B.

In some embodiments described herein, multiple reference nucleic acid sequences may be generated based on one or more target region variants and/or one or more non-target region variants, and the sequence reads may be aligned using the multiple reference nucleic acid sequences. A correct alignment may be determined for a sequence read based on a result of an alignment using the multiple reference nucleic acid sequences. In some embodiments, the multiple reference sequences represent possible combinations of the one or more target region variants and the one or more non-target region variants, and alignment of a sequence read to the multiple reference sequences includes identifying one of the reference sequences that has a series of nucleotides that matches the sequence read.

In some such embodiments, the techniques for analyzing sequencing data may improve the identification of genomic locations of sequence reads, particularly sequence reads associated with homologous regions and/or variants of sequence regions by determining a correct alignment of the sequencing data. The correct alignment may be for a target region, such as a particular gene targeted with a specific primer during sequencing, and/or a non-target region, such as a region that has a level of similarity to a target region that arises from the target region and the non-target region being homologous. Some embodiments include determining the correct alignment by identifying variants for the target region and/or non-target region and generating multiple reference sequences based on the target region variants and the non-target region variants. In some embodiments, the multiple reference sequences may include some or all possible combinations of the target region variants and the non-target region variants. Alignment of the sequence reads may include aligning the sequence reads to the multiple reference sequences to determine a correct alignment. By accounting for different possible variants in the target and non-target regions in the multiple reference sequences, alignment of the sequence reads may include identifying the correct alignment for a sequence read. In some embodiments, alignment of a sequence read may include identifying a particular reference sequence having a region that most closely matches with the sequence read.

Some embodiments relate to generating the multiple reference sequences by combining nucleotide sequences from the sequence reads with one or more archived reference sequences, such as a reference sequence from an archive (e.g., National Center for Biotechnology Information (NCBI) data set). An archived reference sequence may be used as one reference sequence and may be modified to produce one or more other reference sequences, such as modified in a region to include a series of nucleotides that is a known variant of the archived reference in that region. In some embodiments, non-target regions may be identified by aligning some or all of the sequence reads to the archived reference sequence to identify one or more regions to which the sequence reads align. Such regions other than the target region may be identified as non-target region(s). The non-target region(s) and variants of the non-target region(s) may be used in generating the multiple reference sequences. In other embodiments, known homologous or similar regions for a target region may be identified from stored information, such as from an archive (e.g., NCBI) and used in generating reference sequences (together with known variants, in some embodiments).

Described below are examples of systems and methods with which embodiments may operate and techniques that may be implemented in embodiments to configure and operate a sequence analysis system. It should be appreciated, however, that embodiments are not limited to operating in accordance with any of the embodiments below and that other embodiments are possible.

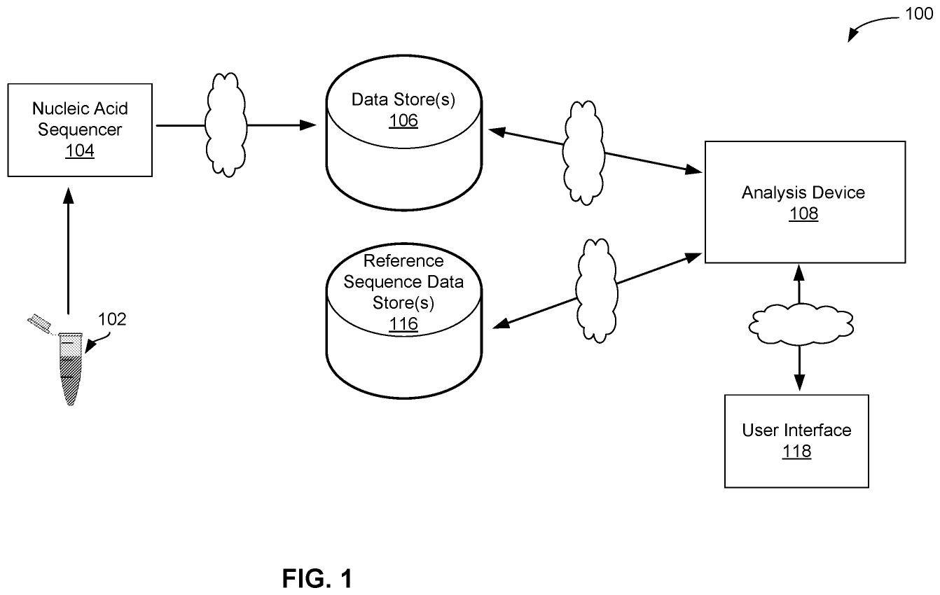

FIG. 1 illustrates a sequence analysis system 100 with which some embodiments may operate. The sequence analysis system 100 of FIG. 1 includes nucleic acid sequencer 104 configured to receive a nucleic acid sample 102 and generate nucleic acid sequencing data for the nucleic acid sample 102. Nucleic acid sample 102 may be prepared in any suitable manner, including extracting and/or isolating nucleic acid sequences from cells or tissue. In some embodiments, nucleic acid sample 102 (e.g., blood sample, biopsy sample, saliva sample) may be obtained from a single organism, such as a single animal, including a single human. The sequencing data may include information identifying multiple nucleic acid sequences, which may be considered as sequence reads. More specifically, the sequencing data may include multiple sequence reads determined from one sample 102. Sequence analysis system 100 includes analysis device 108 configured to analyze the sequencing data, which may include alignment of nucleic acid sequences to one or more reference sequence(s). A result of the analysis may be presented to a user, such as via user interface 118. In some embodiments, the result of the analysis process performed by analysis device 108 on the sequencing data may include information identifying a particular genotype of a biological organism (e.g., human) to which the sample 102 relates. The genotype of the biological organism may identify a type of gene variant present in the organism's genome.

Embodiments are not limited to working with any particular sequencer 104 or type of sequencer 104. Accordingly, nucleic acid sequencer 104 may be configured to perform any suitable type of sequencing process, including sequencing by synthesis, massively parallel sequencing, and Next Generation Sequencing (NGS). The format of sequencing data generated by nucleic acid sequencer 104 may depend on the type of sequencing performed by nucleic acid sequencer 104. In some embodiments, sequencing data may include both information identifying nucleic acid sequences and quality scores associated with the nucleic acid sequences. Sequencing data may have any suitable format, including FASTQ file format and FASTA file format. It should be appreciated that techniques for analyzing sequencing data as described herein are not limited to the type of nucleic acid sequencer used and/or the format of the sequencing data. Nucleic acid sequencer 104 may be a standalone device dedicated to sequencing nucleic acid samples that outputs sequencing data or may be a combination device that may also perform analysis of the sequencing data and may act as analysis device 108.

Results of the sequencing process performed by nucleic acid sequencer 104 may be stored in one or more data store(s) 106. Data store(s) 106 may be configured to store nucleic acid sequencing data generated by nucleic acid sequencer 104. As shown in FIG. 1, data store(s) 106 can be a remote server and nucleic acid sequencer 104 may be configured to transmit sequencing data through one or more wired or wireless networks, including the Internet, to data store(s) 106. In some embodiments, data store(s) 106 may be integrated with or connected to a device that performs nucleic acid sequencing of a sample, such as nucleic sequencer 104. In some embodiments, data store(s) 106 may be a network-attached storage device and sequencing data from nucleic acid sequencer 104 is transmitted over the network(s) to data store(s) 106. In some instances, information identifying nucleic acid sample 102 (e.g., sample ID, primer used to amplify nucleic acid sequences) and/or information identifying the sequencing process used to sequence nucleic acid sample 102 (e.g., type of nucleic acid sequencer used, temperature or timing conditions used during sequencing) may be transmitted with the sequencing data and stored in data store(s) 106 in association with the nucleic acid sequencing data in data store(s) 106. In some embodiments, the additional information may be stored as metadata for a data file containing the nucleic acid sequencing data. The additional information may act to identify the nucleic acid sequencing data, and in some embodiments may be used to retrieve the nucleic acid sequencing data. For example, information identifying a sample ID (e.g., an alphanumeric string) may be used to retrieve nucleic acid sequencing data associated with the sample ID in response to a query for the sample ID. In some embodiments, the additional information may be used to analyze the nucleic acid sequencing data associated with the additional information. For example, the additional information may include information identifying a particular primer used to generate the sample sequenced by nucleic acid sequencer 104. The type of primer and/or the nucleic acid sequence that a primer can amplify may provide an indication of possible target and/or non-target regions of a reference sequence that a sequence read may align to, which the analysis device 108 may use in some embodiments as part of identifying reference sequences.

Analysis device 108 is configured to perform a subsequent analysis on the sequencing data generated by nucleic acid sequencer 104. Analysis device 104 can be a standalone computing device or, in some embodiments, integrated as part of nucleic acid sequencer 104. In some embodiments, analysis device 108 may include a server, such as a remote server accessible over a network. Analysis device 108 may receive nucleic acid sequencing data and/or any additional information associated with the nucleic acid sequencing data from data store(s) 106. Analysis device 108 may receive information identifying one or more reference sequences from one or more reference sequence data store(s) 116. A reference sequence includes one or more series of nucleotides (A, T, C, G). A reference sequence of a particular organism may include one or more series of nucleotides that covers some or all of the genome for the organism. Analysis device 108 may retrieve information identifying one or more reference sequences from reference sequence data store(s) 116 over a network. Reference sequence data store(s) 116 may be associated with a group of people or an organization that maintains and/or updates the reference sequences archived in the reference sequence data store(s) 116. Reference sequence data store(s) 116 may have any suitable form and may include archived reference sequences (e.g., a reference sequence from the National Center for Biotechnology Information (NCBI) archive). Analysis device 108 may align sequence reads from the nucleic acid sequencing data to one or more reference sequences stored in reference sequence data store(s) 116, and/or to one or more reference sequences generated by the analysis device 108, including generated based on reference sequences retrieved from the data store(s) 116. Alignment of a sequence read to a reference sequence by analysis device 108 may indicate a location in the reference sequence matching the sequence read.

In some embodiments, analysis device 108 may retrieve a particular reference sequence from reference sequence data store(s) 116 based on information associated with nucleic acid sequencing data. As an example, information associated with nucleic acid sequencing data may explicitly or implicitly identify a type of organism from which a sample 102 (from which the sequence reads of the sequencing data was determined) was obtained. Analysis device 108 may submit a query to reference sequence data store(s) 116 that includes the information and/or the type of organism and receive in response a reference sequence from reference sequence data store(s) 116 associated with the type of organism. In some embodiments, the reference sequence may be for an entirety of a genome of the type of organism. In other embodiments, the query from the analysis device may include an identification of a target region and/or a non-target region, and one or more reference sequences may be received specific to the region(s). This may be the case where, for example, information associated with sequencing data explicitly identifies a target region or implicitly identifies the target region (and, in some cases, the non-target regions known to be homologous with the target region), such as by identifying a primer that was used to amplify the target region (and that may be known to also amplify one or more non-target regions). In such a case, the analysis device 108 may identify the target and/or non-target regions in the query explicitly or implicitly, such as by identifying the primer.

In some embodiments, analysis device 108 may generate one or more reference sequences, which may be used to analyze nucleic acid sequencing data. Analysis device 108 may be configured to generate one or more reference sequences based on one or more target region variants and/or one or more non-target region variants. In some embodiments, analysis device 108 may generate a reference sequence by modifying an archived reference sequence (e.g., one retrieved from data store 116) to include a series of nucleotides for a target region variant at a location of the target region in the archived reference sequence. In some embodiments, analysis device 108 may additionally or alternatively generate a reference sequence by modifying an archived reference sequence to include a series of nucleotides for a non-target region variant at a location of the non-target region in the archived reference sequence. In some such embodiments, analysis device 108 may generate a reference sequence by modifying an archived reference sequence to include a variant at a location of a target region and a variant at a location of a non-target region. In some cases, the one or more reference sequences generated by analysis device 108 may represent all possible combinations of target region variants and non-target region variants for a group of target region variants and non-target region variants. Any suitable number of reference sequences may be generated as it should be appreciated that the techniques described in the present application are not limited by the number of reference sequences generated. In some embodiments, the number of reference sequences generated may range between 50 to 50,000, or any value or range of values within that range.

Embodiments are not limited to identifying variants, either for a target region or for a non-target region, may be determined in any particular manner. In some embodiments, analysis device 108 may receive user input, such as via user interface 118, identifying a series of nucleotides as a variant for a target region or a non-target region. Analysis device 108 may additionally or alternatively request, from a data store such as reference sequence data store 116, data identifying one or more variants for a target region and/or for a non-target region, and in such a request may identify the target and/or non-target regions. In response, the analysis device 108 may receive data identifying each variant, such as a series of nucleotides that defines a variant for a region and/or a set of “differences” between a standard or average for a region and a variant for that region. In some instances, variant information identifying one or more variants, which may include target region variants and/or non-target region variants, may be stored in association with a reference sequence in a reference sequence data store(s) 116, and analysis device 108 may also receive the variant information in response to submitting a query for the reference sequence and/or may receive the variant information in response to submitting a query that identifies the reference sequence.

In some embodiments, analysis device 108 may identify a series of nucleotides as a variant for a target region and/or a non-target region by aligning one or more sequence reads of a reference sequence, such as an archived reference sequence retrieved from reference sequence data store(s) 116. A location of the reference sequence to which a sequence read aligns may have a particular series of nucleotides, which can be compared to a series of nucleotides of the sequence read (e.g., using the nucleotides of the sequence read and/or complementary nucleotides of the sequence read or using other known alignment techniques) to determine an alignment. If the series of nucleotides at the location of the reference sequence has at least one nucleotide variation in comparison to the series of nucleotides of the sequence read, then the series of nucleotides of the sequence read may be considered as a variant. Depending on whether the location of where the sequence read aligns to the reference sequence is associated with a target region or a non-target region, the variant may be considered as a target region variant or as a non-target region variant. In this manner, analysis device 108 may be configured to determine one or more target region variants and/or one or more non-target region variants according to some embodiments of the present application. Nucleotide sequences associated with these variants may be used to generate reference sequences used in an alignment process of the sequence reads. In some embodiments, a series of nucleotides for a non-target region variant or a target region variant may be identified as having one or more single-nucleotide polymorphisms (SNPs) in comparison to a series of nucleotides for a target region or a non-target region of a reference sequence.

Analysis device 108 may be configured to perform an alignment of one or more sequence reads to multiple reference sequences, which may be generated based on one or more target region variants and one or more non-target region variants. Alignment of a sequence read to one of the reference sequences may include identifying a series of nucleotides of the reference sequence that matches a series of nucleotides identified by the sequence read. In some embodiments, an alignment process performed by analysis device 108 may include comparing a sequence read to each reference sequence of the reference sequences generated by analysis device 108 and identifying a reference sequence that most closely matches the sequence read.

Analysis device 108 may be configured to determine a “correct” alignment for sequence reads for a sample 102 based on a result of the alignment using the reference sequences generated based on one or more target region variants and one or more non-target region variants. An alignment of one sequence read to a reference sequence may be considered as correct when the sequence read matches to a location of the reference sequence with a particular level of accuracy in pairing of the nucleotides of the sequence read to the nucleotides at the location. Determining a correct alignment of a sequence read may include determining a location within a reference sequence that matches to a sequence read above a particular level. In some embodiments, determining the correct alignment includes determining a nucleic acid sequence that aligns to a reference sequence at a sequence location and identifying the nucleic acid sequence as having a target region variant at the sequence location. A correct alignment for sample 102 may be determined from alignment of individual sequence reads.

Analysis device 108 may output an indication of the alignment process to a user, such as via user interface 118. The indication may identify that one or more sequence reads, and/or sample 102, includes a particular target region variant and/or a particular non-target region variant. In some embodiments, the indication may identify a reference sequence to which one or more sequence reads and/or the sample 102 were determined to correctly align, a probability or other metric of confidence indicating how likely the determination of the “correct” match is to be a true match, and/or an identification of and/or probability/metric for any other reference sequences to which the one or more sequence reads and/or may alternatively align. The indication outputted by analysis device 108 may be in any suitable format. In some embodiments, the indication may include information identifying an amount of sequence reads that align to each of the multiple reference sequences generated by analysis device 108, or to at least some of the multiple reference sequences (e.g., a list of the top N aligned reference sequences, where N is some integer less than the number of total reference sequences, such as a top 3, top 5, or top 10 list). In some embodiments, the indication may include information identifying an amount of sequence reads as having each particular target and/or non-target region variant. The amount of sequence reads may include a number, a percentage of total sequence reads, a ratio, or any other suitable measure.

A target region variant identified by an alignment process of analysis device 108 may allow for identification of additional information related to the sequence read. In some embodiments, analysis device 108 may identify an amino acid sequence based on the identified target region variant, and may output an indication of the amino acid sequence. In some embodiments, analysis device 108 may identify a protein associated with an identified amino acid sequence, and may output an indication of the protein. In this manner, analysis device 108 may more accurately determine expression of particular types of proteins by an organism.

The techniques for analyzing nucleic acid sequences described herein may be applied to nucleic acid sequence data associated with any type of biological organism. In some embodiments, the nucleic acid sequence data may be generated using a targeted sequencing method where a primer is used to amplify a particular nucleotide region. A reference sequence used for alignment of the nucleic acid sequence data may be a genome for an organism associated with sequence data. The nucleic acid analysis techniques may be applied to sequencing data associated with one or more regions of the genome that are highly homologous to another region not targeted by the sequencing method. A region may be some or all of a particular gene for the organism. In some embodiments, the nucleic acid sequences may be associated with two or more genes that have similar nucleotide sequences.

In some embodiments, the nucleic acid sequences include human DNA sequences and the reference sequences may include one or more human genome sequences. A human genome sequence may be, for example, a reference human genome from Human Genome Project). The human DNA sequences may include sequences for homologous regions of the human genome. In some embodiments, the human DNA sequences may include sequences for a part or all of a nucleotide coding sequence for a particular gene or set of genes. As examples, the sequences may include nucleotide coding sequences for FC-receptors, immunoglobulin clusters, and telomeres. In some embodiments, analysis of nucleic acid sequences may include identifying an amount of sequence reads corresponding to a particular gene. A genotype for an individual associated with the sequencing data may be determined based on the amount of sequence reads corresponding to the particular gene. In some embodiments, analysis of nucleic acid sequences may include identifying a first amount of sequence reads corresponding to a first gene and a second amount of sequence reads corresponding to a second gene. A nucleotide coding sequence of the first gene and a nucleotide coding sequence of the second gene may have one or more nucleotide variations between the two coding sequences.

Some embodiments relate to analyzing human DNA sequence data associated with targeted sequencing of one or more FC-receptors. In such a case, one or more target regions may include a nucleotide coding sequence for a FC-receptor and one or more non-target regions may include a nucleotide sequence homologous to the nucleotide coding sequence of the FC-receptor. The sequence data may be associated with one or more of the following FC-receptors: FCGR1A, FCGR1B, FCGR1C, FCGR2A, FCGR2B, FCGR2C, FCGR3A, and FCGR3B. In some embodiments, analysis of human DNA sequence data may include identifying a first nucleic acid sequence corresponding to FCGR1A, a second nucleic acid sequence corresponding to FCGR1B, and a third nucleic acid sequence corresponding to FCGR1C. In some embodiments, analysis of human DNA sequence data may include identifying a first nucleic acid sequence corresponding to FCGR2A, a second nucleic acid sequence corresponding to FCGR2B, and a third nucleic acid sequence corresponding to FCGR2C. In some embodiments, analysis of human DNA sequence data may include identifying a first nucleic acid sequence corresponding to FCGR3A and a second nucleic acid sequence corresponding to FCGR3B.

In the context of FC receptors, the FCGR3A and FCGR3B genes are 96.8% identical at the nucleotide level and have transcripts that are 97.4% identical. FCGR3B has at least six nucleotide variants and FCGR3A has at least one nucleotide variant. Some variants for FCGR3B may have more similarity to an archived reference sequence for FCGR3A than to an archived reference sequence for FCGR3B, which may cause sequence reads originating from FCGR2B to align preferentially to the reference FCGR3A sequence over the reference FCGR3B sequence. In addition, the similarity between FCGR3A, FCGR3B, and their respective variants can create challenges in developing primers that preferentially amplify FCGR3A over FCBR3B or vice versa. Techniques described in the present application that include generating multiple reference sequences may allow for determining which gene a particular sequence read aligns to and/or which gene variant corresponds to the nucleotide sequence of the sequence read.

In embodiments where FCGR3A and FCGR3B are targeted, analysis may include modifying 11 or 12 locations of an archived reference sequence with variants to generate at least 4,096 reference sequences according to the techniques described herein. By applying these reference sequences to FCGR3A and FCGR3B targeted sequencing data, individual sequence reads can be determined to correspond to FCGR3A or FCGR3B.

Additional methods for analyzing nucleic acid sequencing data are described below. It should be appreciated that nucleic acid analysis system 100 may be configured to perform any of these methods.

In some embodiments, a sample may be prepared prior to sequencing in a manner that amplifies a particular region of a nucleic acid sequence. In such embodiments, a primer associated with the region may be used during an amplification process to result in preferential sequencing of the region. In this manner, the primer may be considered to amplify a target region of the nucleic acid sequence during an amplification process. In some instances the primer may be complementary to a non-target region of the nucleic acid sequence that is homologous to the target region. The primer may therefore also amplify the non-target region during the amplification process. Such a sequencing process can result in sequence reads corresponding to both the target region and the non-target region.

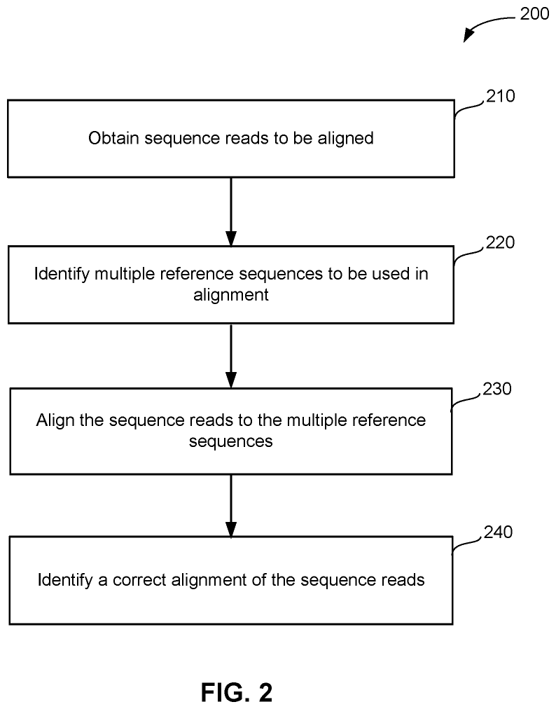

FIG. 2 illustrates an example process 200 that may be implemented in some embodiments to analyze sequencing data using a nucleic acid analysis system, such as system 100 shown in FIG. 1. The process 200 may, in some embodiments, be implemented by an analysis device, like analysis device 108 of FIG. 1.The process 200 begins in block 210, in which the analysis device obtains one or more sequence reads to be aligned. The one or more sequence reads may be obtained by sequencing nucleic acids of a sample, such as by using nucleic acid sequencer 104. As discussed above, data store(s) 106, may store data generated by a nucleic acid sequencer. In some embodiments, the analysis device may obtain sequence reads in block 210 by retrieving one or more sequence reads from the data store(s) 106. The sequence reads may be retrieved by the analysis device using information identifying the one or more sequence reads (e.g., sample ID, primer used in a targeted sequencing process).

In block 220, a reference sequence facility, which may be associated with and executed by the analysis device, identifies multiple reference sequences to be used in alignment of the one or more reference sequences. The reference sequence facility may identify the multiple reference sequences based on a target region and one or more non-target regions, and on one or more target region variants and the one or more non-target region variants. A variant facility, which may be associated with and executed by analysis device 108, may determine the one or more target region variants and the one or more non-target region variants. The variant facility may identify the variants by identifying known variants for the target and/or non-target regions, including by querying a data store (e.g., data store 116) for the known variants. In some embodiments, the reference sequence facility, which may be associated with analysis device 108, may identify multiple reference sequences that are archived in one or more reference sequence data store(s) 116. The archived multiple reference sequences may have been generated based on a prior analyzed sequencing data.

In block 230, a sequence alignment facility triggers an alignment process to determine how sequencing data aligns to the multiple reference sequences to determine a correct alignment. In some embodiments, the sequence alignment facility may perform the alignment process itself, or may initiate an alignment process performed by another device or facility. For example, the sequence alignment facility may transmit to another facility or device (e.g., using interprocess communication, or by sending one or more messages via one or more networks, including the Internet, to one or more other devices) an instruction to initiate an alignment process. In cases in which the alignment is performed by another device or facility, in some cases the sequence alignment facility may communicate the sequence reads and/or the reference sequences to the other facility or device.

Any suitable type of alignment process may be used to align sequencing data to a reference sequence, as embodiments are not limited to implementing any particular type of alignment process. In some embodiments, the alignment process may be adapted particularly for alignment of short sequence reads to a reference sequence. The alignment process may, in some embodiments, include a Burrow-Wheeler transform. Examples of alignment algorithms that may be used during alignment of sequencing data to one or more reference sequences include Bowtie (e.g., Bowtie2 version), BBMap, BWA, BigBWA, BarraCUDA, and CUSHAW.

In block 240, a sequence analysis facility analyzes the sequences to determine a “correct” alignment of the sequencing data to the multiple reference sequences. Any suitable sequence analysis process may be used in identifying variants. In some embodiments, analysis may include using software or an algorithm that generates a data file that includes information of where there are overlapping nucleotides of the sequence reads for each location of a reference sequence. The data file may have any suitable format. An example of software having utilities that may be used as part of analyzing the sequence reads based on the correct alignment may include SAMtools where the command “mpileup” can be used to generate a data file having a pileup format. In some embodiments, an algorithm or software used in sequence analysis may include a single nucleotide polymorphism (SNP) calling function. An example of such software having SNP calling functions is VarScan.

Analysis of sequences to determine the “correct” alignment for a sample and/or sequence reads may include determining an amount of sequence reads, of the sample, that correspond to each of the multiple reference sequences. The amount of sequence reads corresponding to each of the reference sequences may indicate a portion of the sequence reads as having one or more variants, for a target region and/or a non-target region, based on the variant(s) included in each of the reference sequences. In some embodiments, analysis of the sequencing data includes determining a number of sequence reads that aligns to each of the multiple reference sequences. The amount of sequence reads that aligns to each sequence read may indicate whether a particular variant is present among the sequence reads. In some embodiments, identifying the “correct” alignment may include determining a first portion of nucleic acid sequences that align to a first reference sequence of the multiple reference sequences and determining a second portion of nucleic acid sequences that align to a second reference sequence of the multiple reference sequences and identifying which of the first and second reference sequences based on the first portion and the second portion of nucleic acid sequences. Determining the correct alignment may include determine which of the first and second reference sequences align to the highest number of nucleic acid sequences. In some embodiments, determining the “correct” alignment may include determining whether the sequence reads align to the target region or any one non-target region. This may be determined from analysis of the number of sequence reads that align to variants of the target region, and the number of sequence reads that align to variants of each non-target region, and determining the target or non-target region with the highest number of aligned sequence reads.

In some embodiments, one or more amino acid sequences may be determined based on a result of identifying the “correct” alignment of the sequence reads to the multiple reference sequences. The one or more amino acid sequences may be used to identify proteins that an individual associated with the sequencing data has the capacity to express, and these proteins can be used to develop health care personalized to the individual. For a sequence location, the series of nucleotides may vary among the different reference sequences, and the different series of nucleotides may code for different amino acids. Accordingly, the reference sequence that a particular sequence read aligns to may identify an amino acid sequence associated with the sequence read. A result of the correct alignment may allow for determining amino acid sequences associated with different target region variants by identifying a target region variant of the reference sequence a particular sequence read aligns to. In some embodiments, the correct alignment of the sequence reads to the multiple references may allow for determining a first amino acid sequence associated with a first target region variant at a location of a first reference sequence and determining a second amino acid sequence associated with a second target region variant at the same location of a second reference sequence.

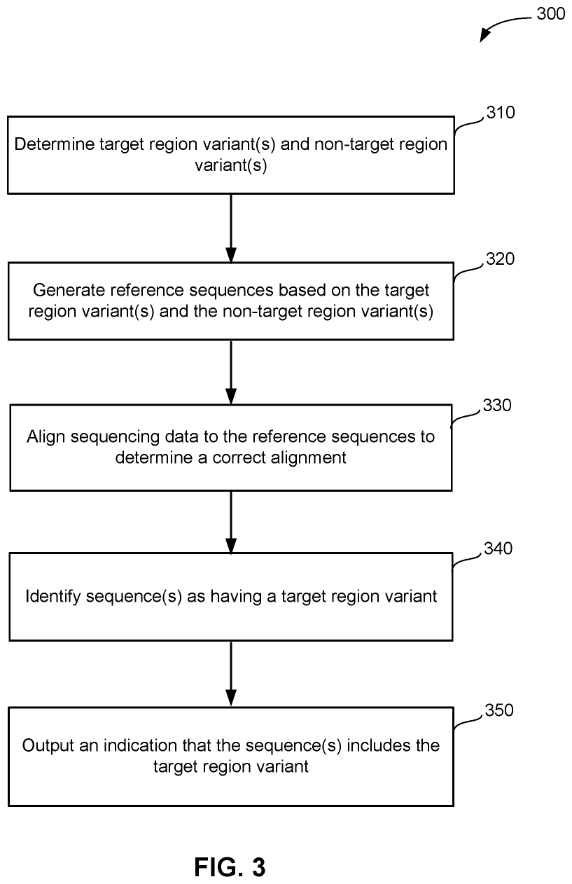

Some embodiments relate to techniques for generating reference sequences based on one or more target region variants and/or one or more non-target region variants and aligning sequencing data to the generated reference sequences. FIG. 3 illustrates an example process 300 that may be implemented by an analysis device in some embodiments to analyze sequencing data using a nucleic acid analysis system, such as system 100 shown in FIG. 1. The process 300 begins in block 310, in which a variant facility determines one or more target region variants and one or more non-target region variants. A target region and a non-target region are at different sequence locations of a genome sequence. Accordingly, a reference sequence may include a series of nucleotides for a target region at a first sequence location and a series of nucleotides for a non-target region at a second sequence location. Determining a target region variant may include determining a series of nucleotides that includes at least one nucleotide variation from the series of nucleotides at the first sequence location. Likewise, determining a non-target region variant may include determining a series of nucleotides that includes at least one nucleotide variation from the series of nucleotides at the second sequence location.

It should be appreciated that the nucleic acid analysis techniques, including generating multiple reference sequences based on target region variant(s) and non-target region variant(s), are not limited to either the number of target regions or the number of non-target regions. Reference sequences according to the techniques described herein may be generated using any suitable number of target regions and any suitable number of non-target regions.

In block 320, a reference sequence facility generates reference sequences based on the one or more target region variants and the one or more non-target region variants. The reference sequences may be generated from a series of nucleotides for a target region, a target region variant, a series of nucleotides for a non-target region, and/or a non-target region variant. In some embodiments, generating the reference sequences may include generating a reference sequence to include, at a first sequence location, a series of nucleotides for a target region or a target region variant and to include, at a second sequence location, a series of nucleotides for a non-target region or a non-target region variant. In other embodiments, a reference sequence may include only nucleotides for a target region or a non-target region, rather than both. In some such embodiments, each reference sequence may include a series of nucleotides for one variant of a target region or a non-target region, with such variants including in some cases the “standard” or “average” series of nucleotides for a region. The reference sequences generated may each be different from one another. In some embodiments, the reference sequences generated may include all possible combinations of target region variants at a first sequence location and non-target region variants at a second sequence location.

Some embodiments relate to generating a reference sequence by modifying an archived reference sequence to include a target region variant and/or a non-target region variant. In some embodiments, generating the reference nucleic acid sequences may include modifying an archived reference sequence at a sequence location to substitute a target region variant for a “standard” or “average” target region of the archived reference sequence. In some embodiments, generating the reference sequences may include modifying an archived reference sequence at a sequence location to substitute a non-target region variant for a “standard” or “average” non-target region of the archived reference sequence. In some embodiments where each reference sequence includes nucleotides for a target region and one or more non-target regions, generating the multiple reference sequences includes generating the multiple reference sequences to have all unique combinations of multiple target region variants at a first sequence location and multiple non-target region variants at a second sequence location.

In block 330, a sequence alignment facility aligns sequencing data to the reference sequences. Aligning the sequencing data to the reference sequences may include determining a correct alignment. A correct alignment of a sequence read to a reference sequence may be defined by one or more parameters of an alignment process. One or more parameters used in aligning the sequencing data may allow for specifying how closely the series of nucleotides for a sequence read matches to a region of a reference sequence for there to be a correct alignment. The one or more parameters may be provided as an input to the alignment process, such as by a user via user interface 118. In some embodiments, one or more parameters of the alignment process may allow for alignment of a sequence read to a reference sequence only when the series of nucleotides of the sequence read exactly match a region of a reference sequence. In such embodiments, the one or more parameters may be considered to have a “very sensitive” setting such that a correct alignment is determined when there is pairing between nucleotides in a sequence read with nucleotides in a reference sequence. In some embodiments, the one or more parameters of the alignment process may allow for alignment of a sequence read to be considered as a correct alignment when there are one or more mismatched nucleotides between the sequence read and a region of a reference sequence. The number of mismatched nucleotides may be less than a threshold number (e.g., 3, 5, 10) and/or less than a threshold percentage (e.g., 1%, 2%, 5%, 10%) of the nucleotides in the read sequence.

In block 340, a sequence analysis facility identifies one or more sequence reads as corresponding to a target region variant. One or more sequences may be identified as having a target region variant based on a reference sequence that the one or more sequences align to and/or a location of a reference sequence that the one or more sequence reads align to. Alignment of a sequence read to a reference sequence may identify the sequence read as having a series of nucleotides, such as a target region variant, included in the reference sequence. In addition, a sequence location of a reference sequence that a sequence read aligns to may identify the sequence read as having a series of nucleotides corresponding to a series of nucleotides at the sequence location. Analyzing a result of the alignment process of act 330 may include identifying, at a target region, which of the multiple reference sequences generate in act 320 have one or more sequence reads that align, and sequence reads may be identified as having a particular target region variant based on which reference sequence the sequence read aligns to. Identifying a target region variant for a particular sequence read may depend on the target region variant at the target region of the reference sequence found to align with the sequence read.

In some embodiments, an amino acid sequence associated with a nucleic acid sequence may be determined based on a nucleic acid sequence for the identified target region variant. The associated amino acid sequence may be determined by identifying a series of amino acids that correspond to the nucleic acid sequence for the identified target region variant. In some embodiments, a protein and/or a protein structure associated with a nucleic acid sequence may be determined based on a nucleic acid sequence for the identified target region. The protein and/or protein structure may be determined based on a series of amino acids that correspond to the nucleic acid sequence for the identified target region variant.

Some embodiments relate to identifying a correct alignment of a sequence read when the sequence read aligns to a region of a reference sequence other than a target region. A non-target region may be determined based on a location in a reference sequence other than a target region to which one or more sequence reads align. During an alignment process, multiple sequence reads may align to one or more non-target regions of the reference sequences. Analyzing a result of the alignment process may include identifying a subset of sequence reads that align to a non-target region, which may include identifying a number of sequence reads that align to different genomic locations of the multiple reference sequences and selecting a genomic location as being a non-target region based on a number of sequence reads that align to that particular genomic location. In some embodiments, a result of the alignment process of act 330 may include a distribution of the number of sequence reads that align to different genomic locations of the reference sequences. Identifying one or more variants of a non-target region may depend on which of the multiple reference sequences that sequence reads align to at the non-target region at a genomic location of the non-target region.

In block 350, a sequence analysis facility outputs an indication that the one or more sequences include one or more determined target region variant. The indication may be presented to a user via a user interface using any suitable format. The indication may include the amount of sequence reads that align with each of the multiple reference sequences. In some embodiments, the indication may include a distribution of the amount of sequence reads that align with each of the multiple reference sequences at a target region where the distribution may include a number, a percentage, a ratio, or another suitable metric for indicating a relative amount of sequence reads associated with each of the multiple reference sequences. As an example, the distribution may be a histogram indicating the number of sequence reads aligning to each reference sequence. The indication may include information identifying multiple sequence reads corresponding to a particular variant at a target region based on which reference sequence the multiple sequence reads align to. In some embodiments, the indication may include information identifying an organism associated with the sequence reads as having a particular genotype at a targeted genomic location based on which of the multiple reference sequences the sequence reads align to and which variants are included in those reference sequences at the targeted genomic location.

In embodiments where an amino acid sequence is determined based on an identified target region variant, outputting an indication may include outputting an indication of the amino acid sequence. A protein may be identified based on the amino acid sequence, and in some embodiments, outputting an indication may include outputting an indication of the protein.

Some embodiments relate to analyzing sequencing data that includes sequence reads originating from more than one gene. Different genes may have nucleotide variation below a threshold amount such that the genes may be considered as highly homologous genes. In some embodiments, the genes may be part of the same family of genes (e.g., FC receptor genes). In some embodiments, two or more genes may have nucleotide sequences that are identical above a threshold percentage (e.g., 85%, 90%, 95%, 96%). In some embodiments, two or more genes may encode for transcripts that are identical above a threshold percentage (e.g., 85%, 90%, 95%, 97%). In some instances, the different genes may have nucleotide variation such that one or more variants of one gene may have a level of similarity with another gene. Such similarity between genes and gene variants may result in misalignment of sequence reads to a reference sequence. In an archived reference sequence, as an example, nucleotide sequences for variant of a first gene may be more similar to a nucleotide sequence for a second gene in the archived reference sequence than to a nucleotide sequence for the first gene in the archived reference sequence. During alignment, a sequence read that includes a series of nucleotides for the variant of the first gene may incorrectly align to the second gene rather than the first gene. Generating reference sequences to include nucleotide sequences for one or more gene variants may reduce or remove the occurrence of this type of misalignment.

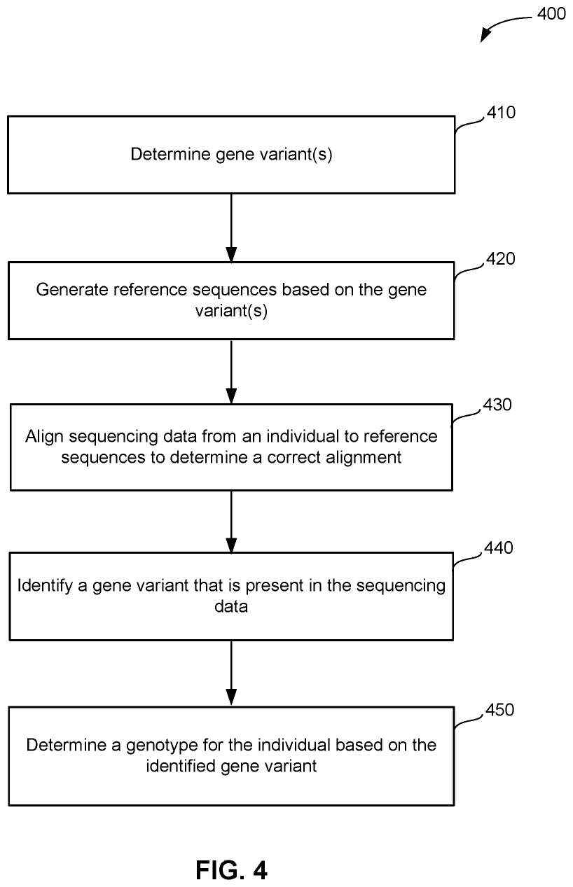

FIG. 4 illustrates an example process 400 that may be implemented in some embodiments for genotyping an individual by analyzing sequencing data associated with the individual. The process 400 begins in block 410, in which a variant facility determines one or more gene variants, which may include variants of the same gene or for multiple different genes. In cases where the one or more gene variants are for different genes, nucleotide sequences and/or transcripts for the different genes may be identical above a threshold amount (e.g., a threshold percentage). In some embodiments, the different genes may include a target gene having a series of nucleotides at a first sequence location and a non-target gene having a series of nucleotides at a second sequence location. Gene variants may be determined by aligning the sequence reads to an archived reference sequence and identifying one or more locations that the sequence reads align to. The one or more locations may include a sequence location for a target gene and/or a sequence location for a non-target gene. A variant for the target gene and/or non-target gene may be determined based on nucleotide variation among the sequence reads. In some embodiments, determining one or more gene variants includes determining at least one first gene variant for a first series of nucleotides of a first gene and at least one second gene variant for a second series of nucleotides of a second gene. A first gene variant may include at least one variation from the first series of nucleotides and at least one variation from the second series of nucleotides. In some embodiments, the first gene is a target gene and the second gene is a non-target gene. The at least one first gene variant may be considered as at least one target gene variant, and the at least one second gene variant may be considered as at least one non-target gene variant.

In block 420, a reference sequence facility generates multiple reference sequences based on the one or more gene variants. A reference sequence may include a nucleotide sequence for a variant of a gene at the genomic sequence location of the geneThe multiple reference sequences may account for variants of multiple genes. As an example, the multiple reference sequences may include variants for a first gene and a second gene where one reference sequence may include a variant of the first gene at a sequence location of the first gene and a variant of the second gene at a sequence location of the second gene.

In block 430, a sequence alignment facility aligns sequencing data from an individual to the multiple reference sequences to determine a correct alignment. The sequencing data may be obtained in any suitable manner. In some embodiments, the sequencing process may include a targeted sequencing process to amplify a particular gene, which may be considered as a target gene, where variants of the target gene are included in the multiple reference sequences. In some embodiments, a targeted sequencing process may generate sequence reads associated with two or more genes where the two or more genes have a level of nucleotide similarity above a threshold. In such embodiments, a correct alignment may include alignment of a first sequence read at a location of a reference sequence associated with a first gene and a second sequence at a location of a reference sequence associated with a second gene. In this manner, use of multiple reference sequences during the alignment process may allow for read sequences to align correctly to sequence locations associated with genes having nucleotide coding sequences that match the read sequences.

In block 440, a sequence analysis facility analyzes the sequences based on the alignment of the sequencing data to the multiple reference sequences to identify a variant for the one or more target genes as being present in the sequencing data. A result of the alignment may indicate a reference sequence that a read sequence align to, and a gene variant included in the reference sequence may be identified as being present in the sequence read.

In block 450, the sequence analysis facility determines a genotype for the individual based on the identified gene variant. Determining the genotype for the individual may be based at least in part on a result of the alignment using the multiple reference sequences. In some embodiments, determining the genotype may include assigning the genotype for the individual based on a reference sequence to which one or more sequence reads align. A gene variant included in the reference sequence may be identified as being present in the individual, and the gene variant may be used to determine a genotype for the individual by identifying the individual as having the gene variant.

In some embodiments, determining a genotype for an individual may include identifying a series of nucleotides associated with a gene or a gene variant that includes at least one variation from the series of nucleotides as being present at a location of the gene in a reference sequence. It should be appreciated that more than one gene can be considered when determining a genotype for an individual. In some embodiments, determining a genotype for an individual may include identifying a first series of nucleotides associated with a first gene or a variant of the first series of nucleotides as being present at a first location and/or a second series of nucleotides associated with a second gene or a variant of the second series of nucleotides as being present at a second location. In these embodiments, the first location and the second location are sequence locations in a reference sequence for the first gene and the second gene, respectively.

Additional information for the sequencing data may be determined based on an identified gene variant. In some embodiments, an amino acid sequence may be determined based on the identified gene variant. In some embodiments, a protein and/or protein structure may be determined based on an amino acid sequence associated with the identified gene variant.

The multiple reference sequences may be used in analysis of sequencing data for one or more other individuals. A genotype for a second individual may be determined by performing an alignment of a second plurality of sequence reads associated with the second individual using the multiple reference sequences. In this manner, the multiple reference sequences may not be generated for each set of sequencing data. Instead, the multiple reference sequences, once determined, may be used for subsequent analysis of sequencing data, which may be obtained using the same or substantially similar sequencing process as the sequencing data used to generate the reference sequences. As an example, a targeted sequencing process may be used to obtain sequencing data for a first individual, and multiple reference sequences may be generated based on the sequencing data. The same targeted sequencing process may be used to obtain sequencing data for a second individual, and the multiple reference sequences may be used in alignment of the sequencing data associated with the second individual. In this manner, the reference sequences may be considered as associated with the targeted sequencing process and may be used in alignment of additional sequencing data obtained by the process.

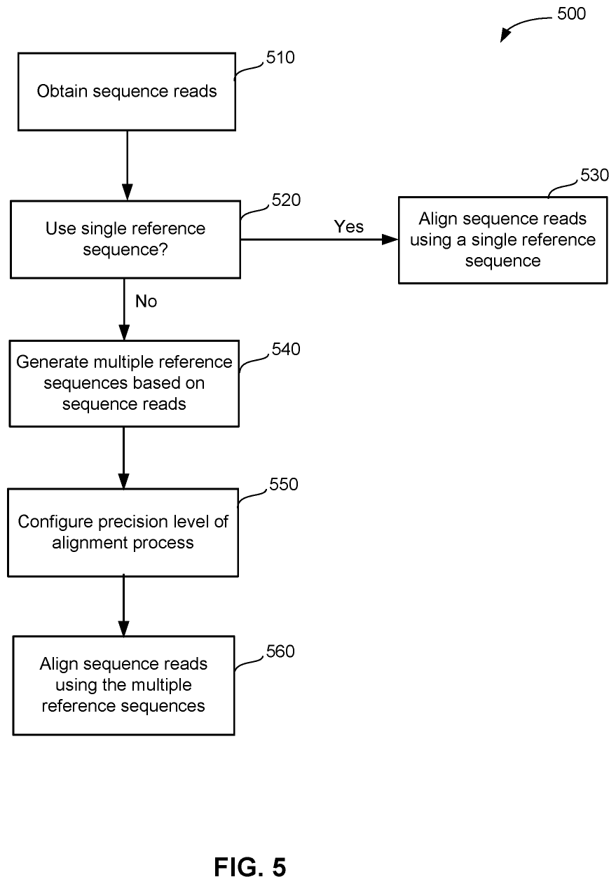

Some embodiments relate to determining whether to use a single (e.g., a single archived) reference sequence in alignment of sequencing data or generate multiple reference sequences based on variants of target and/or non-target regions to use in alignment of sequencing data. FIG. 5 illustrates an example process 500 that may be implemented in some embodiments by an analysis device for analyzing sequencing data by using either a single reference sequence or by generating multiple reference sequences. The process begins at block 510, in which one or more sequence reads to be aligned are obtained. The sequence reads may be obtained using any suitable sequencing process, such as by using nucleic acid sequencer 104.

In block 520, whether to use a single reference sequence in an alignment process of the sequence reads is determined. If the analysis device determines that a single reference sequence is to be used, then process 500 proceeds to block 530, where the sequence reads are aligned using a single reference sequence, which may be a single archived reference sequence. If the outcome of the decision in block 520 is to not use a single reference sequence, then process 500 proceeds to block 540, where a reference sequence facility generates multiple reference sequences based on the sequence reads. The multiple reference sequences may be generated using techniques described herein.

The determination of whether to use a single reference sequence in alignment of the sequence reads may depend on whether a particular target region is homologous with, or otherwise potentially ambiguous with during an alignment process, one or more non-target regions. In some cases, a single reference sequence can be used if sequence reads of a target region are known to align to the single reference sequence with limited or no errors or ambiguities. The single reference sequence may be used alone because, in these cases, the single reference sequence alone may allow for correct alignment of the sequence reads. In such cases, there may be limited value in generating the multiple reference sequences as described herein because an alignment result having few, if any, misalignment of sequence reads to the single reference sequence may be determined using the single reference sequence.

In some cases, determining whether to use a single reference sequence may include performing a preliminary alignment process of the sequence reads. The preliminary alignment process may include aligning the sequence reads to the single reference sequence, and analyzing a result of the preliminary alignment process to identify whether the sequence reads were determined to align to multiple regions (e.g., a target region and one or more non-target regions) based on the sequence locations to which individual sequence reads align. If the sequence reads are found to align entirely or substantially (e.g., more than a certain percentage of sequence reads align, or another threshold analysis) to a target region, then the single reference sequence was sufficient for alignment. In some such cases, results of the preliminary alignment process may be used for subsequent analysis of the sequencing data, without additional alignment. If, however, a substantial number (e.g., more than a certain percentage, or other threshold analysis) of sequence reads are found in the preliminary alignment to align to sequence locations other than a target region, then process 500 may proceed to act 540 of generating multiple reference sequences. In some such cases, the multiple references sequences may be generated based in part on regions to which the sequence reads align in the preliminary alignment, as discussed below.

The determination of whether to use a single reference sequence in alignment of the sequence reads may additionally or alternatively be based on information identifying a type of sample associated with the sequence reads, a type of sequencing process used to obtain the sequence reads, user input, and/or other information that may allow for making this determination. In some embodiments, information used in making this determination may include information identifying that the sequence reads are associated with one, two, or more regions that have nucleotide similarity above a threshold or may otherwise be considered as highly homologous regions. The type of sample and/or the sequencing process used may indicate that the sequence reads are obtained from a targeted sequencing process having a likelihood (e.g., above a threshold) of producing sequence reads corresponding to highly homologous regions. In such a case, if the information associated with the sequence reads and/or the sample indicates that it is unlikely that the sequence reads correspond to highly homologous regions, then process 500 proceeds to block 530, where the single reference sequence is used to align the sequence reads. If the information indicates that it is likely that the sequence reads correspond to highly homologous regions, then process 500 proceeds to block 520, where multiple reference sequences are generated for the highly homologous regions.

In some embodiments, user input may be used to determine whether to use a single reference sequence or not. In the context of system 100 shown in FIG. 1, a user may provide input through user interface 118. The user input may indicate a request, by the user, that an initial alignment or standard alignment be performed for the sequence reads. In some embodiments, an initial alignment of the sequence reads to the single reference sequence may provide an indication of whether further analysis of the sequence reads may be needed, including whether to generate multiple reference sequences and re-align the sequence reads to the generated reference sequences. As an example, an initial alignment of the sequence reads to an archived reference sequence may demonstrate the sequence reads aligning to two or more highly homologous regions of the archived reference sequence. Such an alignment may provide an indication that additional analysis of the sequence reads is required by generating reference sequences based on the sequence reads and aligning the sequence reads to the generated reference sequences.

In some embodiments, user input may indicate a request by the user that there be a level of accuracy in alignment of the sequence reads. If the user input indicates that the level of accuracy in alignment can be below a threshold value, then process 500 may proceed to block 530 where the single reference sequence is used in aligning the sequence reads. If the user input indicates that the level of accuracy in alignment be above a threshold value, then process 500 may proceed to block 540 where multiple reference sequences are generated based on the sequence reads.

In block 550, a precision level of an alignment process to use in aligning the sequence reads is configured. The precision level may determine one or more parameters to be used in alignment of the sequence reads to the multiple generated reference sequences. The one or more parameters may specify how closely the series of nucleotides for a sequence read needs to match to a region of a reference sequence for there to be an alignment. In some embodiments, user input may identify the precision level used in alignment. In some embodiments, a sequence alignment facility may configure the precision level used in alignment based on information stored in association with the sequence reads (e.g., sample ID, type of sequencing process, primer used in amplification). In some embodiments, the precision level may allow that an alignment is identified when nucleotides of a sequence read completely pair with a series of nucleotides of a reference sequence.

In block 550, a sequence alignment facility aligns the sequence reads using the multiple reference sequences. The sequence reads may be aligned using the precision level configured in block 540.

Analysis of sequence reads obtained using a targeted sequencing process may include identifying one or more non-target regions that correspond to a target region associated with the targeted sequencing process and identifying one or more variants of the target region and/or a non-target region. For a particular target region, a region of a sequence other than the target region may be identified as a non-target region when the region is highly homologous to the target region. In addition to identifying one or more non-target regions that corresponds to the target region, a variant of a target region or a non-target region to be included in one of the multiple generated reference sequences may be identified in any suitable manner that allows for identifying nucleotide variation for either the target region or the non-target region. In some embodiments, a non-target region and/or a variant may be identified based on user input. The user input may identify one or more non-target regions corresponding to a particular target region. In some embodiments, the user input may identify one or more variants for a target region and/or a non-target region. In some cases, one or more non-target regions may be identified based on data retrieved from data store(s) storing non-target information identifying one or more non-target regions that correspond to the target region. The non-target information may be stored in association with the information identifying the target region that corresponds to the one or more non-target regions, and the non-target information may be retrieved by querying the data store(s) with the information identifying the target region. Variant information identifying one or more target region variants and/or non-target region variants may be stored in the data store(s). A variant may be identified based on data retrieved from the data store(s) storing the variant information. In such embodiments, the variant information may be stored in association with an archived reference sequence, such as reference sequence data store(s) 116.

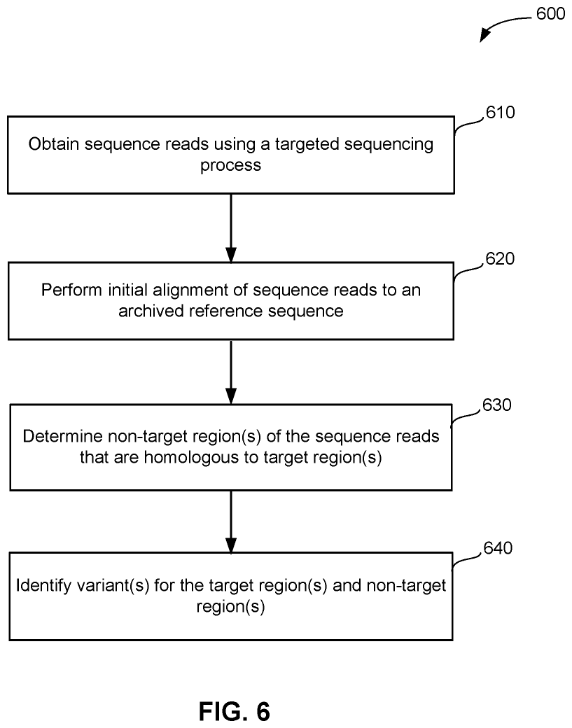

In some embodiments, one or more non-target regions and/or a variant of a target region or a non-target region may be identified based on the sequence reads. In particular, alignment of the sequence reads to a reference sequence (e.g., an archived reference sequence) may be used in identifying one or more non-target regions corresponding to a particular target region and/or one or more variants for a target region and/a non-target region. FIG. 6 illustrates an example process 600 that may be implemented in some embodiments for determining non-target region(s) and identifying variant(s) for a target region and/or a non-target region. The process begins at block 610, in which sequence reads to be aligned are obtained using a targeted sequencing process. The targeted sequencing process may be performed using a primer that amplifies one or more target regions of a nucleic acid sample. In some embodiments, the primer may also amplify an undesired region of the nucleic acid sample, which may be considered as a non-target region.

In block 620, sequence alignment facility performs an initial alignment of the sequence reads to a reference sequence, such as a reference sequence from an archive (e.g., National Center for Biotechnology Information (NCBI) data set). Alignment of the sequence reads to the archived reference sequence may identify one or more sequence locations to which the sequence reads align. The sequence locations may include one or more target regions and/or one or more non-target regions.

In block 630, based om the result of the initial alignment of block 620, a sequence analysis facility determines one or more non-target regions of the sequence reads, which may be homologous to the target region. The one or more non-target regions of the sequence reads may be determined based on one or more sequence locations that the sequence reads align to. A sequence location may correspond to a location within a reference sequence, such as a genome sequence, for a nucleotide coding sequence of a particular gene or set of genes. Alignment of a sequence read to a particular sequence location may identify the sequence read as corresponding to part or all of the nucleotide coding sequence at that sequence location. In some embodiments, the initial alignment may identify a sequence location outside of the sequence location targeted by the sequencing process based on the alignment of one or more sequence reads to the sequence location in the archived reference sequence. The sequence location may be identified, based on the initial alignment, as having a level of similarity or homology with a target region. In this manner, one or more sequence locations may be identified as a non-target region based on where sequence reads align to in the archived reference sequence other than at locations associated with target regions. In some embodiments, a threshold number, percentage, and/or fraction of sequence reads may be used in identifying a sequence location as a non-target region. If the amount of sequence reads that align to a sequence location is above the threshold, then the sequence location may be identified as a non-target region.

In some cases, determining the one or more non-target regions in block 630 may include identifying one or more regions of a genome that are homologous with a target region by identifying one or more regions of a genome that have a degree of similarity to the target region above a threshold amount. Identifying one or more regions of a genome may include identifying one or more regions of the genome that have a degree of similarity to the target region that is higher than a degree of inter-organism variability for the target region.

In block 640, a sequence analysis facility identifies one or more variants for the one or more target regions and the one or more non-target regions. In some embodiments, a variant for a target region and/or a non-target region may be identified based on the alignment of the sequence reads to the archived reference sequence and one or more sequence locations sequence read aligns. In some embodiments, a variant for a non-target region may be identified based on a sequence location associated with the non-target region. Variant information associated with the sequence location may be retrieved, such as from a reference sequence data store or archive, and used to identify variants for the non-target region. A variant of a target region and/or a non-target region may be incorporated into a reference sequence used in alignment of sequence reads, particularly sequence reads that may misalign with an archived reference sequence (e.g., National Center for Biotechnology Information (NCBI) data set).

Multiple reference sequences may be generated by incorporating different combinations of variants into individual reference sequences such that the variation of the multiple reference sequences may be representative of different possible combinations of the variants. Using these reference sequences for alignment of the sequence reads may improve alignment.

In some cases, reference sequences for alignment of sequence reads may be generated using information retrieved from an archive. Such an archive may identify, for example, regions that are known to be homologous with one another. Rather than performing a preliminary alignment to identify non-target regions that may be homologous with a target region, in some embodiments, based on information identifying the target region, information on non-target regions may be retrieved from an archive. In a similar manner, information on variants of target and/or non-target regions may also be retrieved. This information on regions and variants may then be used to generate multiple reference sequences in some embodiments.

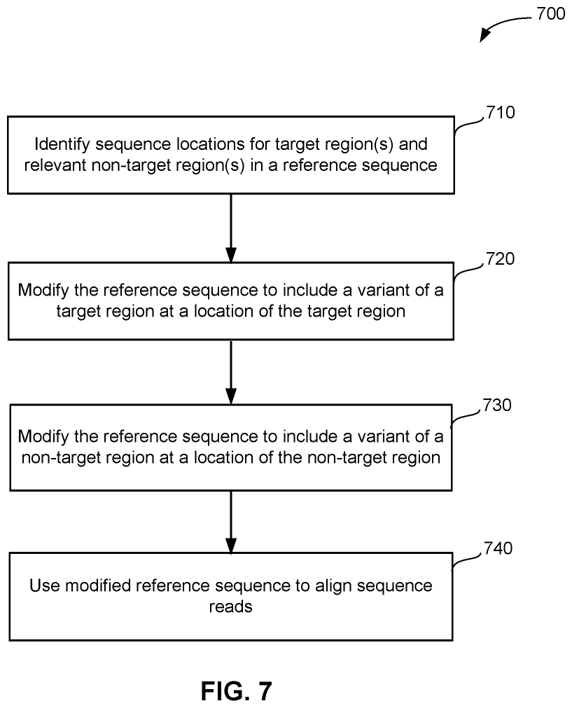

FIG. 7 illustrates an example process 700 that may be implemented in some embodiments for generating multiple reference sequences for a target region and one or more non-target regions, and/or one or more variants for those regions. The process begins at block 710, in which a facility identifies one or more sequence locations for one or more target regions. The target region may be input to the facility or determined by the facility, such as by being included in or determined from information on a sample or on a primer used in processing a sample as discussed above, or based on other information. The facility may then identify one or more non-target regions that are known to be homologous to the target region. The non-target regions may be determined by the facility, for example, by querying a data store of information regarding such regions, including a data store of information on known homologous regions. The result of the query may include a listing of one or more non-target regions (or none, if none are known) that are known to be homologous. Such a listing of known homologous regions may have been input to the data store in any suitable manner, including through a manual input, as embodiments are not limited in this respect. In addition, the facility may query the data store for information

In block 720, a reference sequence facility begins generating reference sequences for use in alignment. To do so, the facility may modify a reference sequence one or more times and thus generate one or more reference sequences that each include a variant of the target region at the location of the target region.

In block 730, a reference sequence facility additionally generates reference sequences for use in alignment using the one or more non-target regions. For example, the facility may modify a reference sequence that includes a non-target region one or more times to include, in each generated reference sequence, a variant of a non-target region at a location of that non-target region. This may be repeated for each non-target region, and for each variant of each non-target region.

In block 740, a sequence alignment facility uses the generated reference sequences to align sequence reads. Alignment of the sequence reads may include identifying, for a sequence read, a reference sequence that most closely matches as having a region that matches the sequence read.

Information based on aligning sequence reads to multiple reference sequences may be output having any suitable format to a user, such as via user interface 118. In some embodiments, the output may identify a reference sequence to which one or more sequence reads determined to correctly align, a probability or other metric of confidence indicating how likely the determination of the “correct” match is to be a true match, and/or an identification of and/or probability/metric for any other reference sequences to which the one or more sequence reads and/or may alternatively align. In some embodiments, an amount of sequence reads that aligns to each reference sequence may be included as an output, or to at least some of the multiple reference sequences (e.g., a list of the top N aligned reference sequences, where Nis some integer less than the number of total reference sequences, such as a top 3, top 5, or top 10 list). In some embodiments, the indication may include information identifying an amount of sequence reads as having each particular target and/or non-target region variant. The amount of sequence reads may include a number, a percentage of total sequence reads, a ratio, or any other suitable measure.

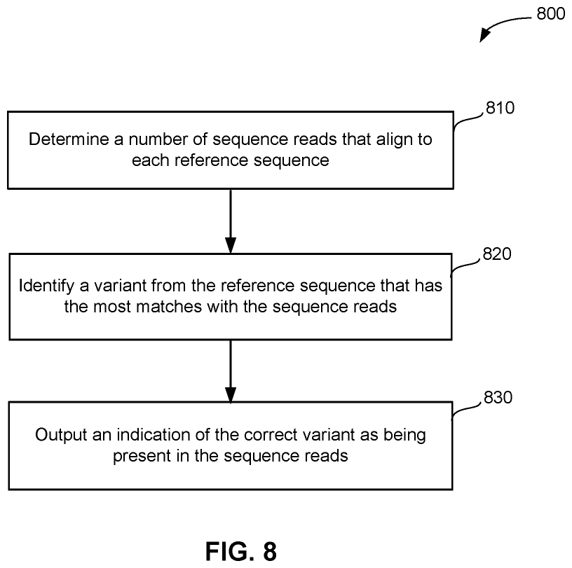

The output may include information identifying multiple sequence reads corresponding to a particular variant at a target region based on which reference sequence the multiple sequence reads align to. In some embodiments, the indication may include information identifying an organism associated with the sequence reads as having a particular genotype at a targeted genomic location based on which of the multiple reference sequences the sequence reads align to and which variants are included in those reference sequences at the targeted genomic location. In embodiments where an amino acid sequence is determined based on an identified target region variant, and the output may include an indication of the amino acid sequence. A protein may be identified based on the amino acid sequence, and in some embodiments, the output may include an indication of the protein. Depending on the amount of sequence reads for a particular reference sequence, a variant included in the reference sequence may be identified as a correct variant as present in the sequence reads. FIG. 8 illustrates an example process 800 that may be implemented in some embodiments for identifying a variant as being present in a sequence read. The process begins at block 810, in which a sequence analysis facility determines a number of sequence reads that align to each reference sequence. In some embodiments, the number of sequence reads that align to each reference sequence includes only those sequence reads that exactly match at the nucleotide level to a region of a reference sequence. In some embodiments, a number of sequence reads that align to a region of a reference sequence with one or more nucleotide mismatches may be determined. The number of nucleotide mismatches may be below a threshold value. The number of nucleotide mismatches may be determined based on user input and/or one or more parameters used during an alignment process. In some embodiments, a number of sequence reads that exactly match a region of a reference sequence and a number of sequence reads that match with one or more nucleotide variations at the region of the reference sequence may be determined. In block 820, a sequence analysis facility identifies a variant from the reference sequence that has the most matches with the sequence reads. The sequence reads that match at a region of a reference sequence may identify a location of the reference sequence and a variant included at the location of the reference sequence. The number of sequence reads that match with a variant of a reference sequence may identify the variant as having the most matches with the sequence reads. In some embodiments, identifying a variant from a reference sequence that has the most matches with the sequence reads may include identifying a target region or a non-target region that includes the variant.

In block 830, sequence analysis facility outputs an indication of the “correct” variant as being present in the sequence reads. The “correct” variant may be the variant identified as having the most matches with the sequence reads. The “correct” variant may be used to identify a genotype of an individual associated with the sequence reads. In some embodiments, the indication may include a number of variants identified as being present in the sequence reads based on the number of sequence reads that align to each reference sequence.

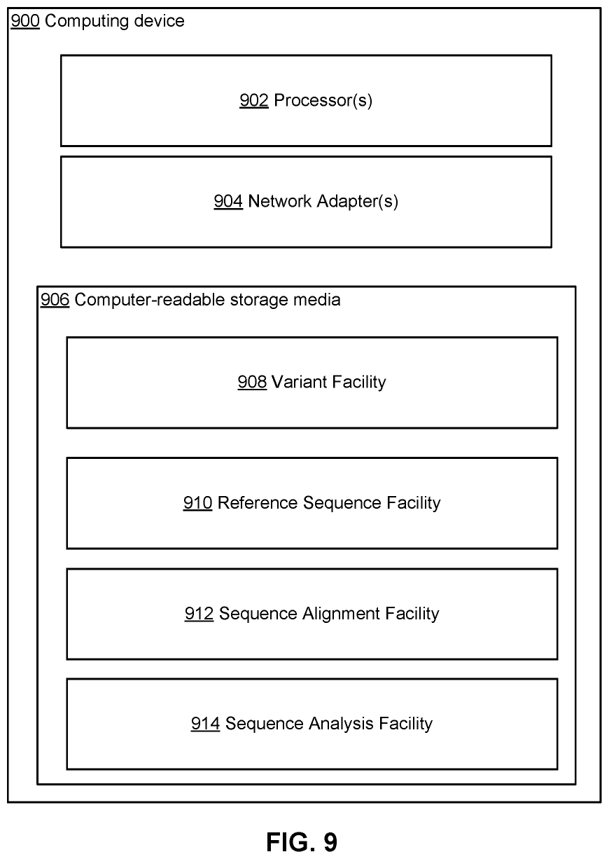

FIG. 9 illustrates one exemplary implementation of a computing device in the form of a computing device 900 that may be used in a system implementing techniques described herein, although others are possible. Computing device 900 may operate a sequence analysis device and control the functionality of the sequence analysis device using hardware, software or a combination thereof. When implemented in software, the software code can be executed on any suitable processor or collection of processors, whether provided in a single component or distributed among multiple components. Such processors may be implemented as integrated circuits, with one or more processors in an integrated circuit component. A processor may be implemented using circuitry in any suitable format. Computing device 400 may be integrated within the sequence analysis device or operate the sequence analysis device remotely. It should be appreciated that FIG. 9 is intended neither to be a depiction of necessary components for a computing device to operate in accordance with the principles described herein, nor a comprehensive depiction.

Computing device 900 may comprise at least one processor 902, a network adapter 904, and computer-readable storage media 906. Computing device 900 may be, for example, a desktop or laptop personal computer, a personal digital assistant (PDA), a smart mobile phone, a tablet computer, a server, or any other suitable portable, mobile or fixed computing device. Network adapter 904 may be any suitable hardware and/or software to enable the computing device 900 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media 906 may be adapted to store data to be processed and/or instructions to be executed by processor 902. Processor 902 enables processing of data and execution of instructions. The data and instructions may be stored on the computer-readable storage media 906 and may, for example, enable communication between components of the computing device 900.

The data and instructions stored on computer-readable storage media 906 may comprise computer-executable instructions implementing techniques which operate according to the principles described herein. In the example of FIG. 9, computer-readable storage media 906 stores computer-executable instructions implementing various facilities and storing various information as described above. Computer-readable storage media 906 may store a variant facility 908, a reference sequence facility 910, a sequence alignment facility 912, and a sequence analysis facility 914 each of which may implement techniques described above.

While not illustrated in FIG. 9 computing device 900 may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format, through visible gestures, through haptic input (e.g., including vibrations, tactile and/or other forces), or any combination thereof.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

One or more processors may be interconnected by one or more networks in any suitable form, including as a local area network or a wide area network, such as an enterprise network or the Internet. Such networks may be based on any suitable technology and may operate according to any suitable protocol and may include wireless networks, wired networks, or fiber optic networks.

One or more algorithms for controlling methods or processes provided herein may be embodied as a readable storage medium (or multiple readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible storage medium) encoded with one or more programs that, when executed on one or more computers or other processors, perform methods that implement the various methods or processes described herein.

In some embodiments, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the methods or processes described herein. As used herein, the term “computer-readable storage medium” encompasses only a computer-readable medium that can be considered to be a manufacture (e.g., article of manufacture) or a machine. Alternatively or additionally, methods or processes described herein may be embodied as a computer readable medium other than a computer-readable storage medium, such as a propagating signal.

The terms “program” or “software” are used herein in a generic sense to refer to any type of code or set of executable instructions that can be employed to program a computer or other processor to implement various aspects of the methods or processes described herein. Additionally, it should be appreciated that according to one aspect of this embodiment, one or more programs that when executed perform a method or process described herein need not reside on a single computer or processor, but may be distributed in a modular fashion amongst a number of different computers or processors to implement various procedures or operations.

Executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. Non-limiting examples of data storage include structured, unstructured, localized, distributed, short-term and/or long term storage. Non-limiting examples of protocols that can be used for communicating data include proprietary and/or industry standard protocols (e.g., HTTP, HTML, XML, JSON, SQL, web services, text, spreadsheets, etc., or any combination thereof). For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags, or other mechanisms that establish relationship between data elements.

The phraseology and terminology used herein is for the purpose of description and should not be regarded as limiting. The use of “including,” “comprising,” “having,” “containing,” “involving,” and variations thereof, is meant to encompass the items listed thereafter and additional items. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

What is claimed:

1. A system comprising:
a nucleic acid sequencer;
a nucleic acid analysis device;
an alignment device, the alignment device configured to:
receive a plurality of nucleic acid sequences from the nucleic acid sequencer;
determine a correct alignment of a plurality of nucleic acid sequences, wherein the correct alignment is a target region having a first series of nucleotides at a first sequence location or at least one non-target region having at least one second series of nucleotides at least one second sequence location, wherein determining the correct alignment comprises:
determining at least one target region variant for the first series of nucleotides and at least one non-target region variant for the at least one second series of nucleotides, wherein each of the at least one target region variant includes at least one variation from the first series of nucleotides and each of the at least one non-target region variant includes at least one variation from one of the at least one second series of nucleotides,
generating a plurality of reference nucleic acid sequences based on the at least one target region variant and the at least one non-target region variant,
performing an alignment of the plurality of nucleic acid sequences using the plurality of reference nucleic acid sequences, and
determining the correct alignment for the plurality of nucleic acid sequences based at least in part on a result of the alignment using the plurality of reference nucleic acid sequences; and
provide the correct alignment for the plurality of nucleic acid sequences to the nucleic acid alignment device.

2. The system of claim 1, wherein generating the plurality of reference nucleic acid sequences comprises generating a plurality of reference nucleic acid sequences from the first series of nucleotides for the target region, the at least one target region variant, the at least one second series of nucleotides for the at least one non-target region, and the at least one non-target region variant.

3. The system of claim 2, wherein:
the at least one second sequence location is a second sequence location;
the at least one second series of nucleotides is a second series of nucleotides; and
generating the plurality of reference nucleic acid sequences comprises generating a plurality of sequences including, at the first sequence location, one of the first series of nucleotides or the at least one target region variant and including, at the second sequence location, one of the second series of nucleotides or the at least one non-target region variant, each sequence of the plurality of sequences being different.

4. The system of claim 1, wherein determining the correct alignment further comprises:
determining the at least one non-target region, wherein determining the at least one non-target region comprises analyzing an alignment of at least a subset of the plurality of nucleic acid sequences to a reference sequence to identify regions to which the at least the subset align.

5. The system of claim 4, wherein generating the plurality of reference nucleic acid sequences comprises generating a first reference nucleic acid sequence, of the plurality, by modifying the reference sequence at the first sequence location to substitute one target region variant, of the at least one target region variant, for the target region of the reference sequence.

6. The system of claim 4, wherein generating the plurality of reference nucleic acid sequences comprises generating a second reference nucleic acid sequence, of the plurality, by modifying the reference sequence at the second sequence location to substitute one non-target region variant, of the at least one non-target region variant, for the non-target region of the reference sequence.

7. The system of claim 4, wherein the plurality of nucleic acid sequences comprise human DNA and the reference sequence is a human genome sequence.

8. The system of claim 1, wherein the alignment device is further configured to:
determine the at least one non-target region based on the target region, wherein determining the at least one non-target region comprises identifying one or more regions of a genome that are homologous with the target region.

9. The system of claim 8, wherein identifying one or more regions of a genome that are homologous with the target region comprises identifying one or more regions of a genome that have a degree of similarity to the target region above a threshold.

10. The system of claim 9, wherein identifying one or more regions of a genome that have a degree of similarity to the target region above a threshold comprises identifying one or more regions of the genome that have a degree of similarity to the target region that is higher than a degree of inter-organism variability for the target region.

11. The system of claim 1, wherein determining the correct alignment comprises:
determining a first nucleic acid sequence of the plurality of nucleic acid sequences that aligns to a first reference sequence of the plurality of reference sequences at least at the first sequence location,
identifying the first nucleic acid sequence as having a target region variant of the at least one target region variant at the first sequence location of the first reference sequence, and
outputting an indication that the first nucleic acid sequence includes the target region variant.

12. The system of claim 11, wherein the alignment device is further configured to determine an amino acid sequence associated with the first nucleic acid sequence based on a nucleic acid sequence for the target region variant.

13. The system of claim 12, wherein outputting the indication that the first nucleic acid sequence includes the target region variant comprises outputting an indication of the amino acid sequence.

14. The system of claim 13, wherein outputting the indication that the first nucleic acid sequence includes the target region variant comprises outputting an indication of a protein associated with the amino acid sequence.

15. The system of claim 1, wherein determining the correct alignment comprises determining a first portion of the plurality of nucleic acid sequences that align to a first reference sequence of the plurality of reference sequences and determining a second portion of the plurality of nucleic acid sequences that align to a second reference sequence of the plurality of reference sequences.

16. The system of claim 15, wherein the method further comprises determining a first amino acid sequence associated with a first target region variant at the first location of the first reference sequence and a second amino acid sequence associated with a second target region variant at the first location of the second reference sequence.

17. The system of claim 1, wherein determining the correct alignment comprises determining an amount of nucleic acid sequences of the plurality of nucleic acid sequences that align with each of the plurality of reference sequences.

18. The system of claim 1, wherein:
determining the correct alignment comprises determining a reference sequence of the plurality of reference sequences that the nucleic acid sequence aligns to and identifying a series of nucleotides at the first location in the reference sequence; and
the nucleic acid analysis device is configured to assign a genotype for an individual associated with a nucleic acid sequence of the plurality of nucleic acid sequences based on the reference sequence of the plurality of reference sequences to which the nucleic acid sequence aligns.

19. The system of claim 1, wherein:
the at least one target region variant includes a plurality of target region variants and the at least one non-target region variant includes a plurality of non-target region variants, and
generating the plurality of reference nucleic acid sequences further comprises generating the plurality of reference nucleic acid sequences to have all unique combinations of the plurality of target region variants at the first sequence location and the plurality of non-target region variants at the second sequence location.

20. The system of claim 1, wherein the target region includes at least a portion of a first gene and the non-target region includes at least a portion of a second gene.

21. The system of claim 1, wherein the sequence data is human DNA sequence data, the at least one target region includes a nucleotide coding sequence for a FC-receptor, and the at least one non-target region includes a nucleotide sequence homologous to the nucleotide coding sequence.

22. The system of claim 21, wherein the FC-receptor is selected from the group consisting of FCGR1A, FCGR1B, FCGR1C, FCGR2A, FCGR2B, FCGR2C, FCGR3A, and FCGR3B.

23. The system of claim 21, wherein the method further comprises identifying a first nucleic acid sequence of the plurality of nucleic acid sequences corresponding to FCGR3A and a second nucleic acid sequence of the plurality of nucleic acid sequences corresponding to FCGR3B.

24. The system of claim 1, wherein the nucleic acid sequencer is coupled to the alignment device, and the alignment device is coupled to the nucleic acid analysis device.

25. The system of claim 1, wherein identifying the non-target region having the second series of nucleotides at the second sequence location further comprises identifying the second series of nucleotides as having at least one single-nucleotide polymorphism in comparison to the first series of nucleotides at the first location.

26. The system of claim 1 wherein the nucleic acid analysis device is configured to:
determine a genotype for the individual from the plurality of nucleic acid sequences, wherein the plurality of nucleic acid sequences are associated with the individual.

27. The system of claim 26, wherein the nucleic acid analysis device is further configured to determine an amino acid sequence based on the identified variant.

28. The system of claim 27, wherein the nucleic acid analysis device is further configured to determine a protein structure based on the amino acid sequence.

29. The system of claim 26, wherein the nucleic acid analysis device is further configured to:
determine a genotype for a second individual by performing an alignment of a second plurality of nucleic acid sequences associated with the second individual using the plurality of reference nucleic acid sequences to identify the first series of nucleotides or one of the at least one first gene variant as being present at the first location and/or the second series of nucleotides or one of the at least one second gene variant as being present at the second location.

30. A method of analyzing sequencing data, the method comprising:
determining a correct alignment of a plurality of nucleic acid sequences, wherein the correct alignment is a target region having a first series of nucleotides at a first sequence location or at least one non-target region having at least one second series of nucleotides at at least one second sequence location, wherein determining the correct alignment comprises:
determining at least one target region variant for the first series of nucleotides and at least one non-target region variant for the at least one second series of nucleotides, wherein each of the at least one target region variant includes at least one variation from the first series of nucleotides and each of the at least one non-target region variant includes at least one variation from one of the at least one second series of nucleotides;
generating a plurality of reference nucleic acid sequences based on the at least one target region variant and the at least one non-target region variant;
performing an alignment of the plurality of nucleic acid sequences using the plurality of reference nucleic acid sequences; and
determining the correct alignment for the plurality of nucleic acid sequences based at least in part on a result of the alignment using the plurality of reference nucleic acid sequences.

31. The method of claim 30, wherein generating the plurality of reference nucleic acid sequences comprises generating a plurality of reference nucleic acid sequences from the first series of nucleotides for the target region, the at least one target region variant, the at least one second series of nucleotides for the at least one non-target region, and the at least one non-target region variant.

32. The method of claim 31, wherein:
the at least one second sequence location is a second sequence location;
the at least one second series of nucleotides is a second series of nucleotides; and
generating the plurality of reference nucleic acid sequences comprises generating a plurality of sequences including, at the first sequence location, one of the first series of nucleotides or the at least one target region variant and including, at the second sequence location, one of the second series of nucleotides or the at least one non-target region variant, each sequence of the plurality of sequences being different.

33. The method of claim 30, wherein determining the correct alignment further comprises:
determining the at least one non-target region, wherein determining the at least one non-target region comprises analyzing an alignment of at least a subset of the plurality of nucleic acid sequences to a reference sequence to identify regions to which the at least the subset align.

34. The method of claim 33, wherein generating the plurality of reference nucleic acid sequences comprises generating a first reference nucleic acid sequence, of the plurality, by modifying the reference sequence at the first sequence location to substitute one target region variant, of the at least one target region variant, for the target region of the reference sequence.

35. The method of claim 33, wherein generating the plurality of reference nucleic acid sequences comprises generating a second reference nucleic acid sequence, of the plurality, by modifying the reference sequence at the second sequence location to substitute one non-target region variant, of the at least one non-target region variant, for the non-target region of the reference sequence.

36. The method of claim 33, wherein the plurality of nucleic acid sequences comprise human DNA and the reference sequence is a human genome sequence.

37. The method of claim 30, further comprising:
determining the at least one non-target region based on the target region, wherein determining the at least one non-target region comprises identifying one or more regions of a genome that are homologous with the target region.

38. The method of claim 37, wherein identifying one or more regions of a genome that are homologous with the target region comprises identifying one or more regions of a genome that have a degree of similarity to the target region above a threshold.

39. The method of claim 38, wherein identifying one or more regions of a genome that have a degree of similarity to the target region above a threshold comprises identifying one or more regions of the genome that have a degree of similarity to the target region that is higher than a degree of inter-organism variability for the target region.

40. The method of claim 30, wherein determining the correct alignment comprises:
determining a first nucleic acid sequence of the plurality of nucleic acid sequences that aligns to a first reference sequence of the plurality of reference sequences at least at the first sequence location,
identifying the first nucleic acid sequence as having a target region variant of the at least one target region variant at the first sequence location of the first reference sequence, and
outputting an indication that the first nucleic acid sequence includes the target region variant.

41. The method of claim 40, wherein the method further comprises determining an amino acid sequence associated with the first nucleic acid sequence based on a nucleic acid sequence for the target region variant.

42. The method of claim 41, wherein outputting the indication that the first nucleic acid sequence includes the target region variant comprises outputting an indication of the amino acid sequence.

43. The method of claim 42, wherein outputting the indication that the first nucleic acid sequence includes the target region variant comprises outputting an indication of a protein associated with the amino acid sequence.

44. The method of claim 30, wherein determining the correct alignment comprises determining a first portion of the plurality of nucleic acid sequences that align to a first reference sequence of the plurality of reference sequences and determining a second portion of the plurality of nucleic acid sequences that align to a second reference sequence of the plurality of reference sequences.

45. The method of claim 44, wherein the method further comprises determining a first amino acid sequence associated with a first target region variant at the first location of the first reference sequence and a second amino acid sequence associated with a second target region variant at the first location of the second reference sequence.

46. The method of claim 30, wherein determining the correct alignment comprises determining an amount of nucleic acid sequences of the plurality of nucleic acid sequences that align with each of the plurality of reference sequences.

47. The method of claim 30, wherein:
determining the correct alignment comprises determining a reference sequence of the plurality of reference sequences that the nucleic acid sequence aligns to and identifying a series of nucleotides at the first location in the reference sequence; and
the method further comprises assigning a genotype for an individual associated with a nucleic acid sequence of the plurality of nucleic acid sequences based on the reference sequence of the plurality of reference sequences to which the nucleic acid sequence aligns.

48. The method of claim 30, wherein:
the at least one target region variant includes a plurality of target region variants and the at least one non-target region variant includes a plurality of non-target region variants, and
generating the plurality of reference nucleic acid sequences further comprises generating the plurality of reference nucleic acid sequences to have all unique combinations of the plurality of target region variants at the first sequence location and the plurality of non-target region variants at the second sequence location.

49. The method of claim 30, wherein the target region includes at least a portion of a first gene and the non-target region includes at least a portion of a second gene.

50. The method of claim 30, wherein the sequence data is human DNA sequence data, the at least one target region includes a nucleotide coding sequence for a FC-receptor, and the at least one non-target region includes a nucleotide sequence homologous to the nucleotide coding sequence.

51. The method of claim 50, wherein the FC-receptor is selected from the group consisting of FCGR1A, FCGR1B, FCGR1C, FCGR2A, FCGR2B, FCGR2C, FCGR3A, and FCGR3B.

52. The method of claim 50, wherein the method further comprises identifying a first nucleic acid sequence of the plurality of nucleic acid sequences corresponding to FCGR3A and a second nucleic acid sequence of the plurality of nucleic acid sequences corresponding to FCGR3B.

53. The method of claim 30, wherein identifying the non-target region having the second series of nucleotides at the second sequence location further comprises identifying the second series of nucleotides as having at least one single-nucleotide polymorphism in comparison to the first series of nucleotides at the first location.

54. At least one computer-readable storage medium storing computer-executable instructions that, when executed, perform a method of analyzing sequence data, the method comprising:
determining a correct alignment of a plurality of nucleic acid sequences, wherein the correct alignment is a target region having a first series of nucleotides at a first sequence location or at least one non-target region having at least one second series of nucleotides at at least one second sequence location, wherein determining the correct alignment comprises:
determining at least one target region variant for the first series of nucleotides and at least one non-target region variant for the at least one second series of nucleotides, wherein each of the at least one target region variant includes at least one variation from the first series of nucleotides and each of the at least one non-target region variant includes at least one variation from one of the at least one second series of nucleotides;
generating a plurality of reference nucleic acid sequences based on the at least one target region variant and the at least one non-target region variant;
performing an alignment of the plurality of nucleic acid sequences using the plurality of reference nucleic acid sequences; and
determining the correct alignment for the plurality of nucleic acid sequences based at least in part on a result of the alignment using the plurality of reference nucleic acid sequences.

55. An apparatus comprising:
control circuitry configured to:
determine a correct alignment of a plurality of nucleic acid sequences, wherein the correct alignment is a target region having a first series of nucleotides at a first sequence location or at least one non-target region having at least one second series of nucleotides at least one second sequence location, wherein determining the correct alignment comprises:
determining at least one target region variant for the first series of nucleotides and at least one non-target region variant for the at least one second series of nucleotides, wherein each of the at least one target region variant includes at least one variation from the first series of nucleotides and each of the at least one non-target region variant includes at least one variation from one of the at least one second series of nucleotides;
generating a plurality of reference nucleic acid sequences based on the at least one target region variant and the at least one non-target region variant;
performing an alignment of the plurality of nucleic acid sequences using the plurality of reference nucleic acid sequences; and
determining the correct alignment for the plurality of nucleic acid sequences based at least in part on a result of the alignment using the plurality of reference nucleic acid sequences.

56. A method for genotyping an individual, the method comprising:
determining a genotype for the individual from a plurality of nucleic acid sequences associated with the individual, wherein the genotype is based on a first gene at a first sequence location or a second gene at a second sequence location, and wherein determining the genotype comprises:
determining at least one first gene variant for a first series of nucleotides associated with the first gene and at least one second gene variant for a second series of nucleotides associated with the second gene, wherein the first series of nucleotides includes at least one variation from the second series of nucleotides, and wherein each of the at least one first gene variant includes at least one variation from the first series of nucleotides and each of the at least one second gene variant includes at least one variation from one of the second series of nucleotides;
generating a plurality of reference nucleic acid sequences based on the at least one first gene variant and the at least one second gene variant;
performing an alignment of the plurality of nucleic acid sequences using the plurality of reference nucleic acid sequences; and
determining the genotype for the individual based at least in part on a result of the alignment using the plurality of reference nucleic acid sequences to identify the first series of nucleotides or one of the at least one first gene variant as being present at the first location and/or the second series of nucleotides or one of the at least one second gene variant as being present at the second location.

57. The method of claim 56, wherein the method further comprises determining an amino acid sequence based on the identified variant.

58. The method of claim 57, wherein the method further comprises determining a protein structure based on the amino acid sequence.

59. The method of claim 56, wherein the method further comprises:
determining a genotype for a second individual by performing an alignment of a second plurality of nucleic acid sequences associated with the second individual using the plurality of reference nucleic acid sequences to identify the first series of nucleotides or one of the at least one first gene variant as being present at the first location and/or the second series of nucleotides or one of the at least one second gene variant as being present at the second location.
